(19) United States
(12) Patent Application Publication
(10) Pub. No.: US 2003/0144350 A1
(43) Pub. Date: Jul. 31, 2003

(54) Fat accumulation-modulation compounds

(75) Inventors: Michael John Stevenson, Haverhill, MA (US); Harrison Jefferson Leighton, Boston, MA (US)

Correspondence Address: LAHIVE & COCKFIELD, 28 STATE STREET, BOSTON, MA, 02109, US

(73) Assignee: AdipoGenix, Inc., Boston, MA

(21) Appl. No.: 10/201,588

(22) Filed: Jul. 22, 2002

Related U.S. Application Data
(60) Provisional application No. 60/306,837, filed on Jul. 20, 2001.

(51) Int. Cl. A61K 31/325; A61K 31/20; A61K 31/18; A61K 31/165

(52) U.S. Cl. 514/485; 514/602; 514/558; 514/617

(57) ABSTRACT

The present invention pertains to compounds effective at modulating fatty acid or triglyceride (“fat”) accumulation by cells, such compounds having therapeutic potential as regulators of body mass and for the treatment of overweight individuals, obesity, and metabolic disorders. Featured compounds are set forth and exemplified herein. Therapeutic methods and pharmaceutical compositions featuring these compounds are also provided.

### RELATED APPLICATIONS

This application claims the priority of U.S. provisional patent application no. 60/306,837, filed Jul. 20, 2001, incorporated herein by reference.

### GOVERNMENT RIGHTS

This invention was made at least in part with support under grant number R43DK54588, entitled “Antiobesity Drug Development Using Human Preadipocytes,” awarded by the United States National Institutes of Health.

### BACKGROUND OF THE INVENTION

In the past few decades, we have seen a great increase in the prevalence of obesity in both the Western world and in developing third world countries. Obesity has recently been declared by The World Health Organization (WHO) as a global epidemic that “pose[s] one of the greatest threats to human health and well being.”

In the United States, it is estimated that at least half of all Americans over the age of 20 are overweight and that 20% of men and 25% of women are clinically obese (BMI or body mass index >30). In the United Kingdom it is estimated that 17% of men and 20% of women in England and Wales are obese. The prevalence of overweight individuals and obesity is also increasing in other countries including Southeast Asia, Latin America, and the Middle East. (see Dove (2001) Nature Biotechnol. 19:25-28.) Moreover, the WHO indicates that obesity is increasing globally.

Obesity greatly increases the risk of premature death as well as specific diseases including but not limited to hypertension, Type 2 diabetes, cardiovascular disease or morbidity, respiratory problems, and a number of cancers. Obesity and overweight individuals are also taking a significant financial toll on developed and developing nations. The National Institute of Medicine (NIM) “estimates that the direct health care costs and loss of productivity resulting from ill health costs the United States more than $70 billion a year” (Dove, supra).

Despite the ever increasing prevalence of overweight individuals and obesity, there have been only limited advances in pharmaceutical therapies for the treatment of these disorders. Anti-obesity drugs have been marketed or are currently being developed that target a host of biochemical mechanisms involved in regulating eating behavior, fat metabolism, and energy expenditure.

Fenfluramine, phentermine, and dexfenfluramine are or were market drugs aimed at centrally suppressing appetite. Dexfenfluramine, marketed under the name Redux™, was withdrawn from the market in 1997 due to cases of valvular heart damage in subjects taking Redux. A drug called Orlistat, marketed under the name Xenical™, is a lipase inhibitor that targets the breakdown and absorption of ingested dietary fats; however, side effects of Xenical™, which have deterred many subjects from therapy with this drug, include gas, increased bowel movements, an urgent need to have them and an inability to control them.

Recently, a great deal of excitement has centered on drugs aimed at increasing energy expenditure by enhancing thermogenesis (or heat production) by cells. Thyroid hormone receptors and β-adregenergic receptors have been targeted and many such drugs are either marketed or in advanced clinical trials; however, drugs targeting thyroid hormones, for example, have been linked to detrimental side effects such as loss of bone calcium. Another mechanistic target are the uncoupling proteins (UCPs), i.e., proteins that uncouple respiration and shunt energy from metabolic pathways to heat generation. This effective “wasting” of energy results in cells having to utilize more stored fat to maintain normal cellular functions.

Recently, an exciting cerulenin analog, C75, has been tested as a potential anti-obesity therapeutic in mice. (Loftus et al. (2001) Science 288: 2379). Laboratory mice injected with C75 lost profound amounts of weight. Mice that received single injections of C75 ate 90 percent less than what their untreated littermates consumed. The researchers also administered C75 to a strain of genetically obese mice. These mice, too, lost tremendous amounts of weight. Further studies in fasting animals suggest a role for yet another molecule, namely an appetite-stimulating molecule found in the hypothalamus called “neuropeptide Y.”

Given the ever increasing prevalence of obesity and overweight individuals globally, and the relatively limited choices of clinically effective therapeutics unhampered by detrimental or undesirable side effects, there exists a need to develop a wider range of pharmaceuticals, in particular, compounds that target varying mechanisms of appetite regulation, fat metabolism, or energy expenditure.

### SUMMARY OF INVENTION

The present invention pertains to compounds effective at modulating fatty acid or triglyceride (“fat”) accumulation by cells, such compounds having therapeutic potential as regulators of body mass and for the treatment of overweight individuals, obesity, and metabolic disorders. The present invention features compounds and methods of modulating the accumulation of fatty acids or triglycerides by fat cells, e.g. adipocytes or preadipocytes, in vivo or in vitro.

Featured compounds are those corresponding to the Formulae set forth herein. Preferred compounds are depicted in the Examples and Drawings. Therapeutic methods and pharmaceutical compositions (e.g. drugs or prodrugs) featuring these compounds are also provided.

Preferred compounds of the invention have some of the following exemplary chemical substructures:

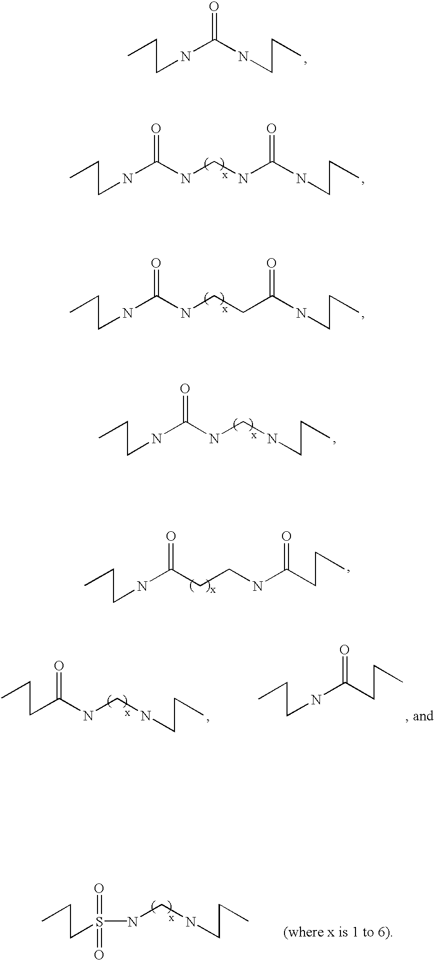

### BRIEF DESCRIPTION OF THE DRAWINGS

FIGS. 1-11 list the chemical structures of some exemplary inhibitors of fatty acid accumulation of the invention.

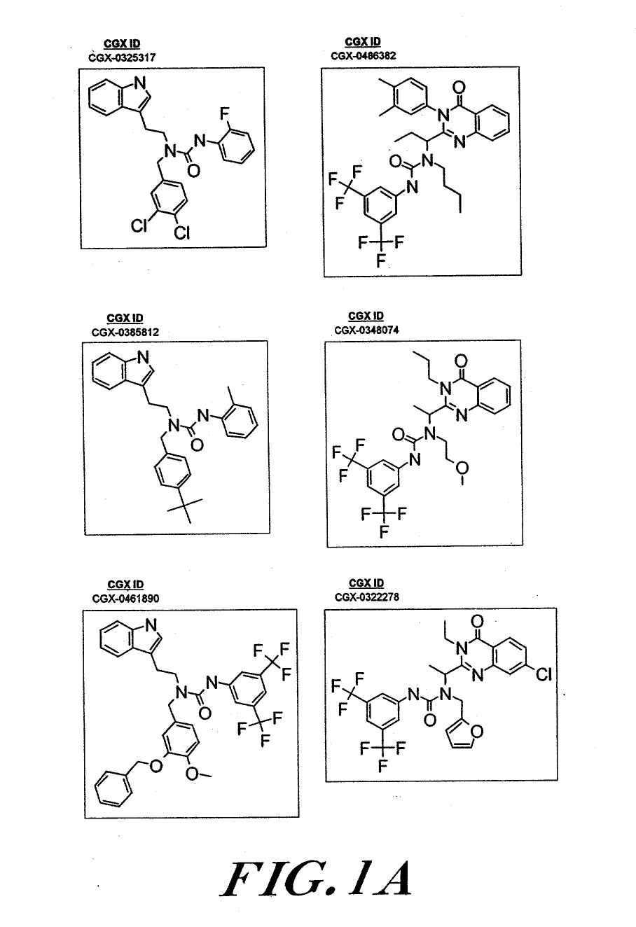

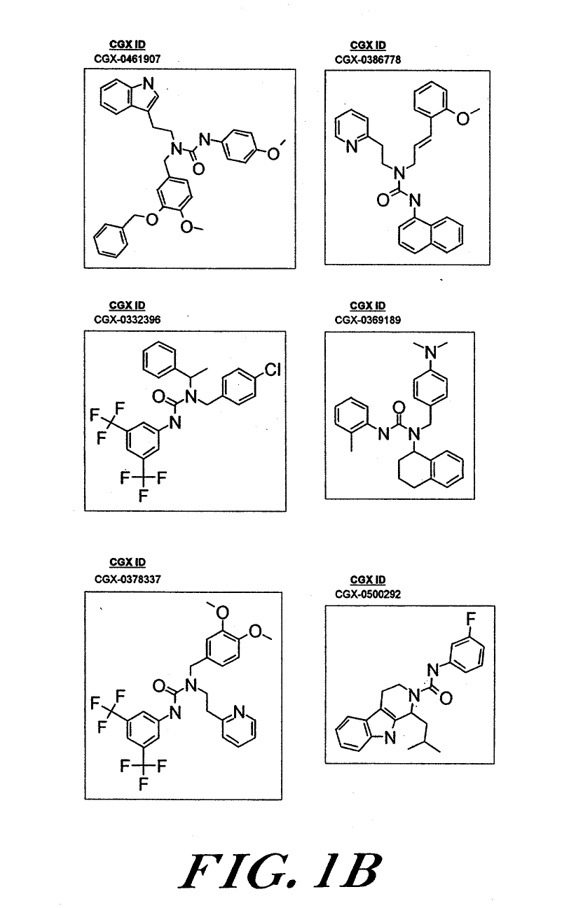

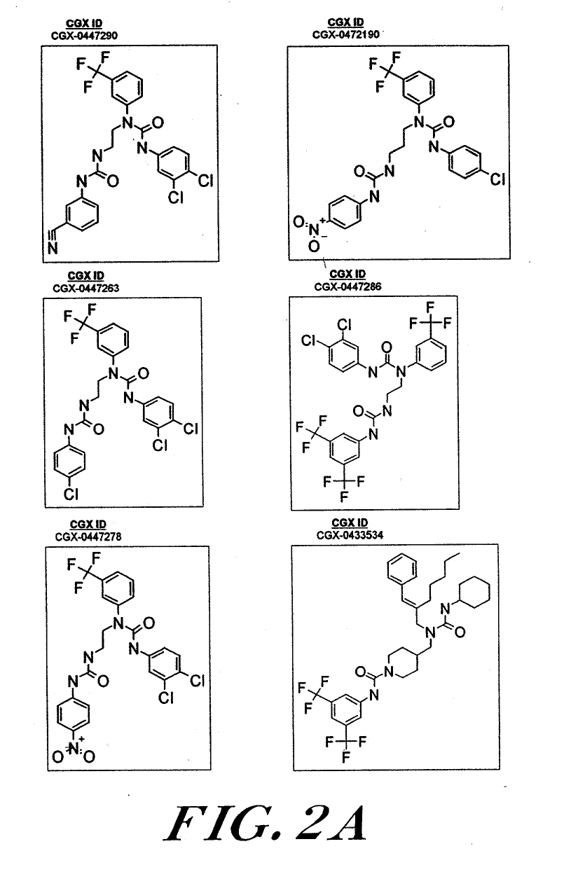

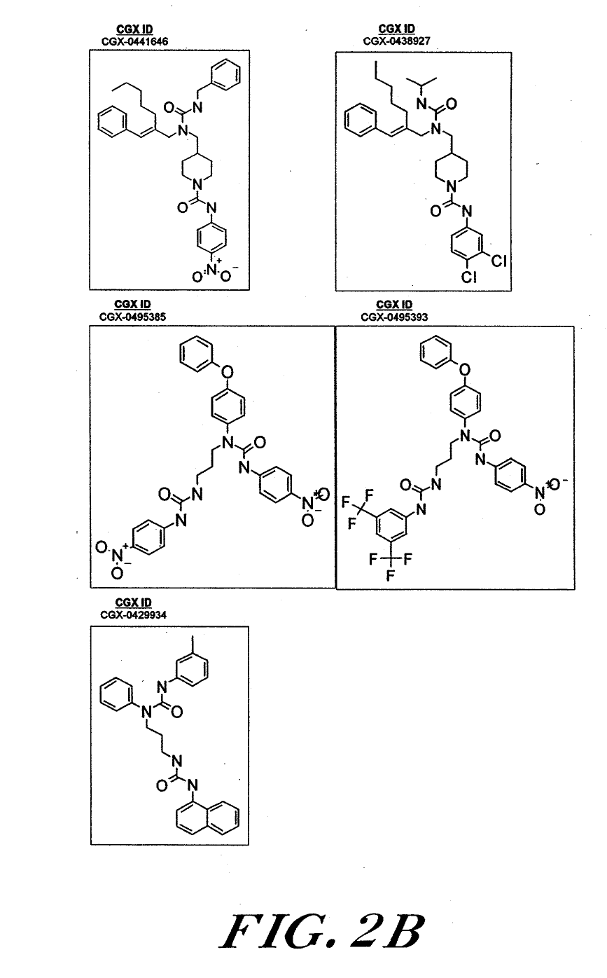

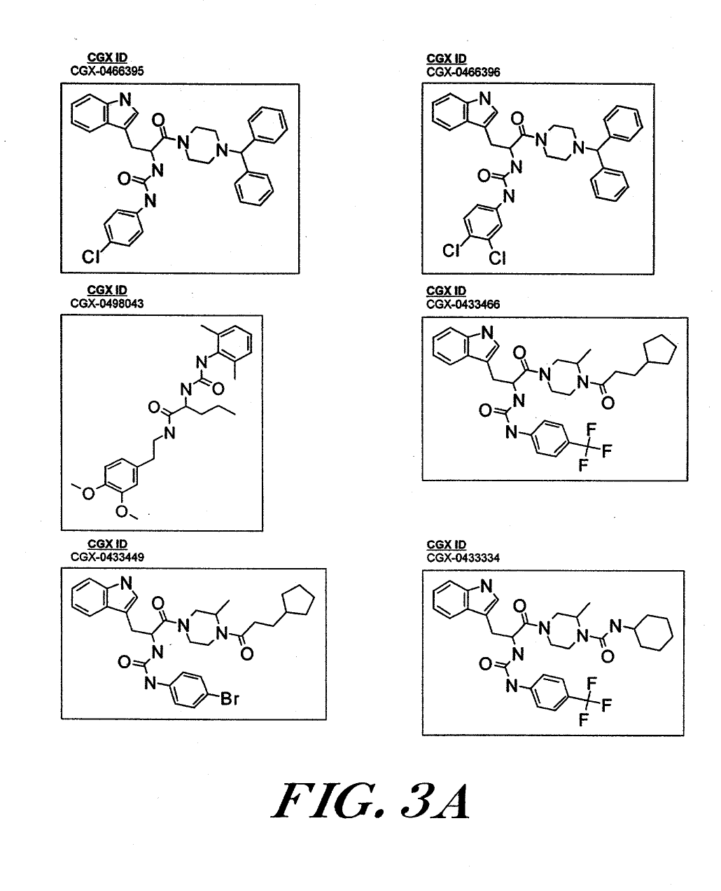

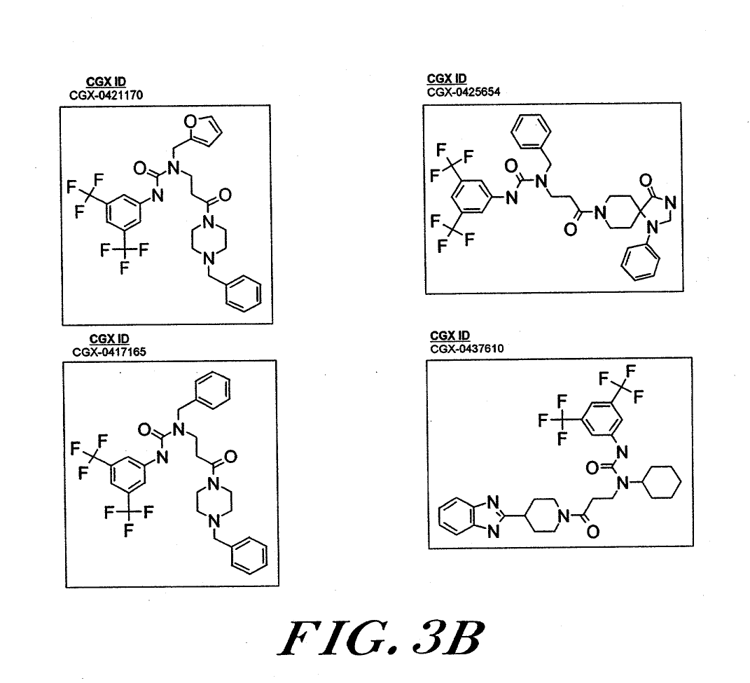

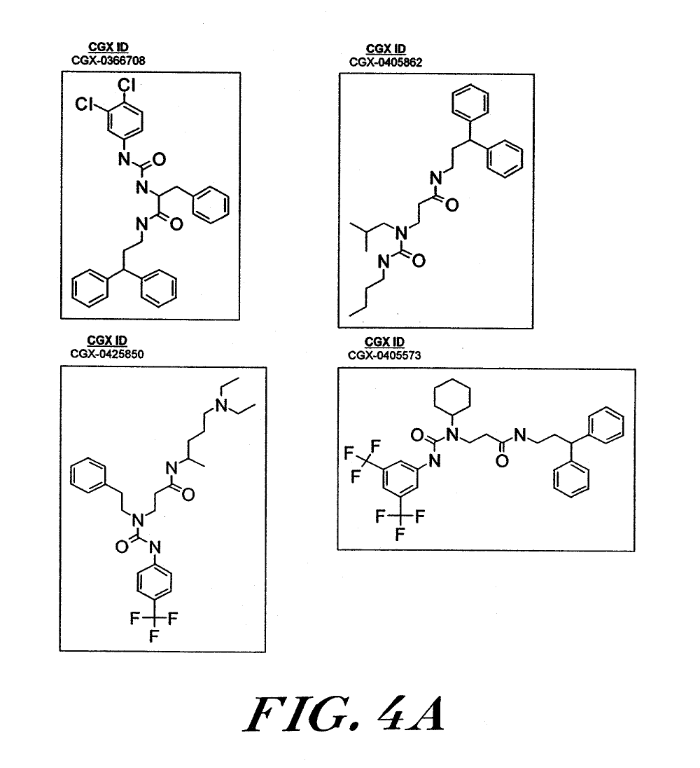

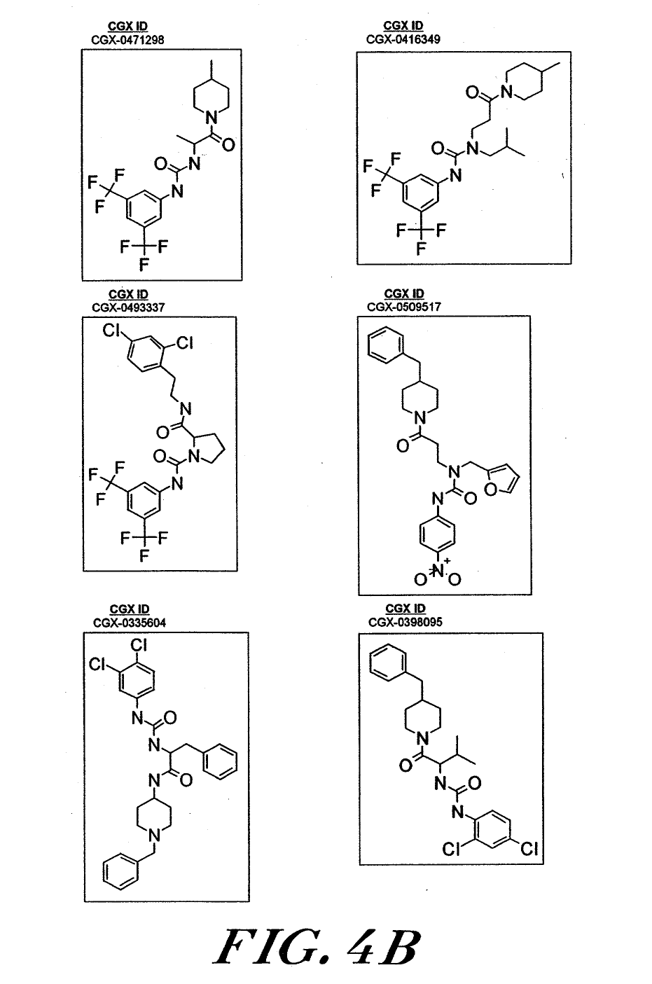

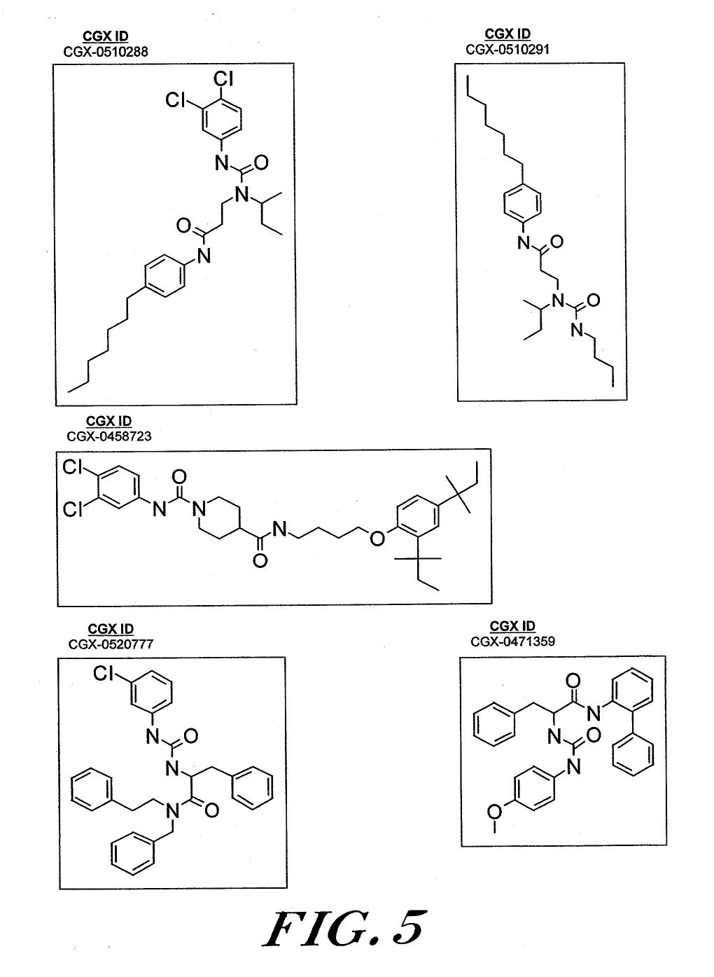

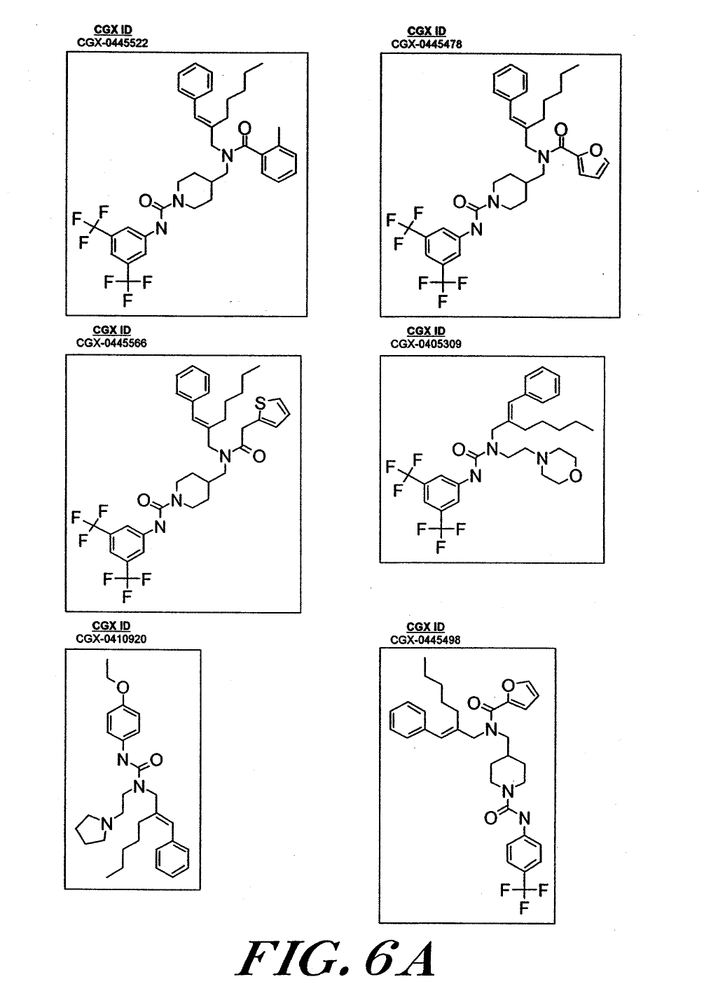

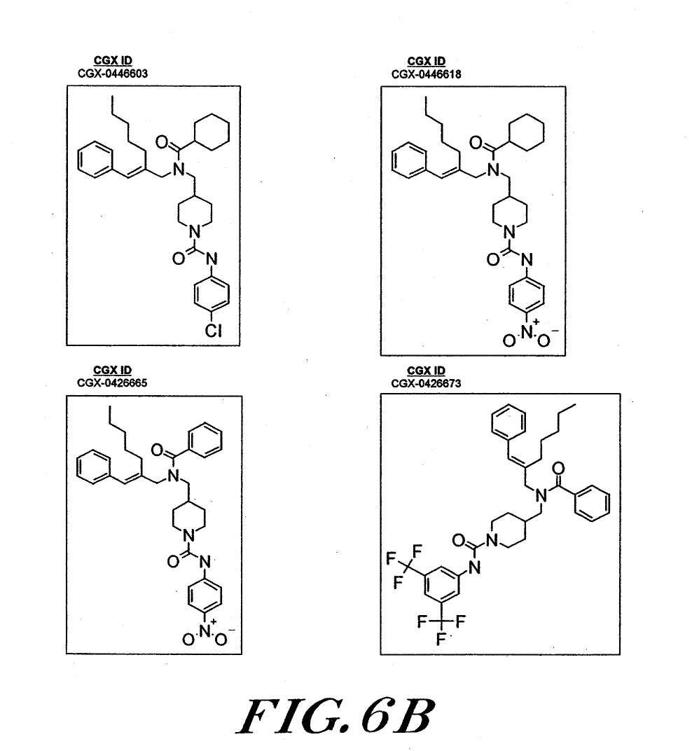

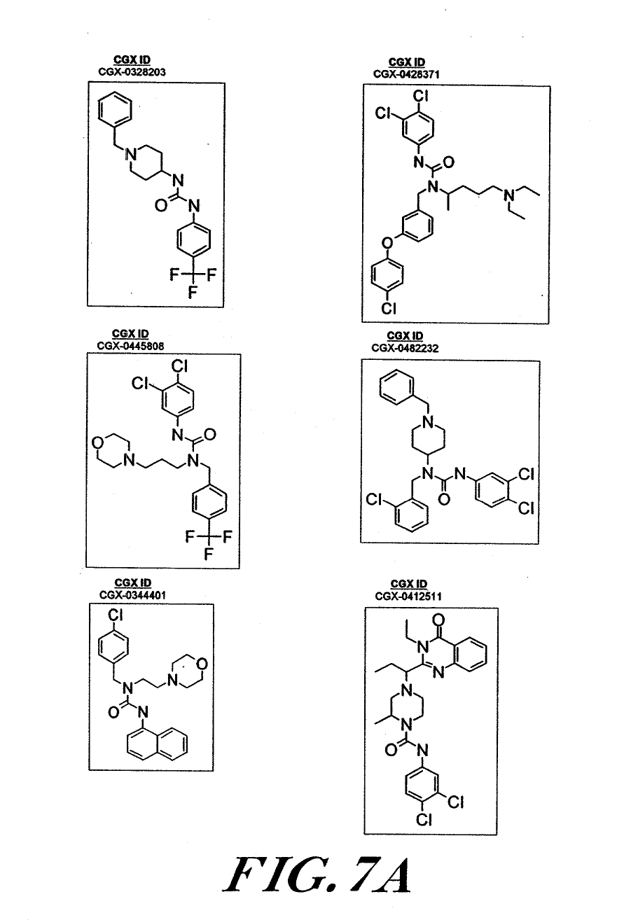

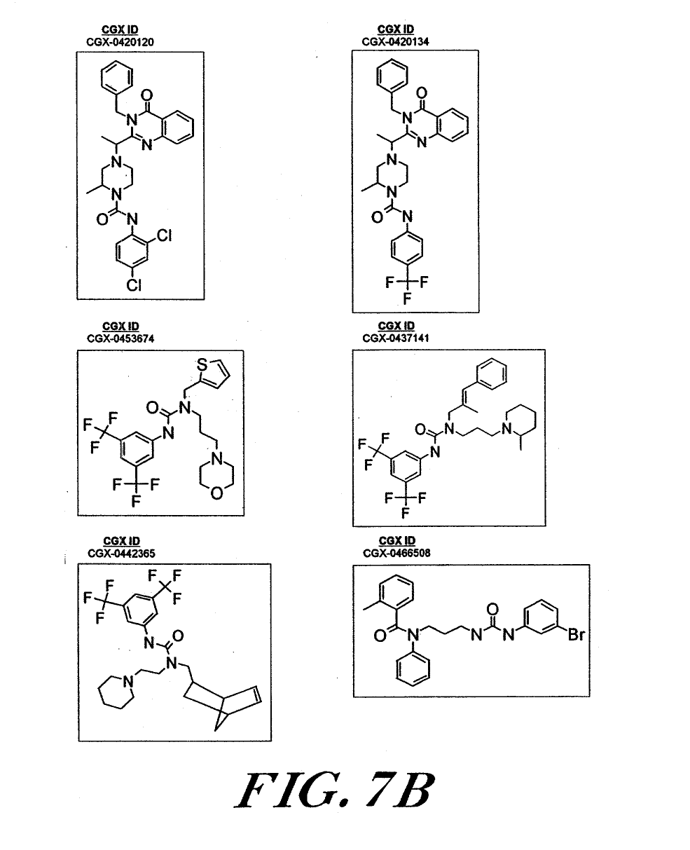

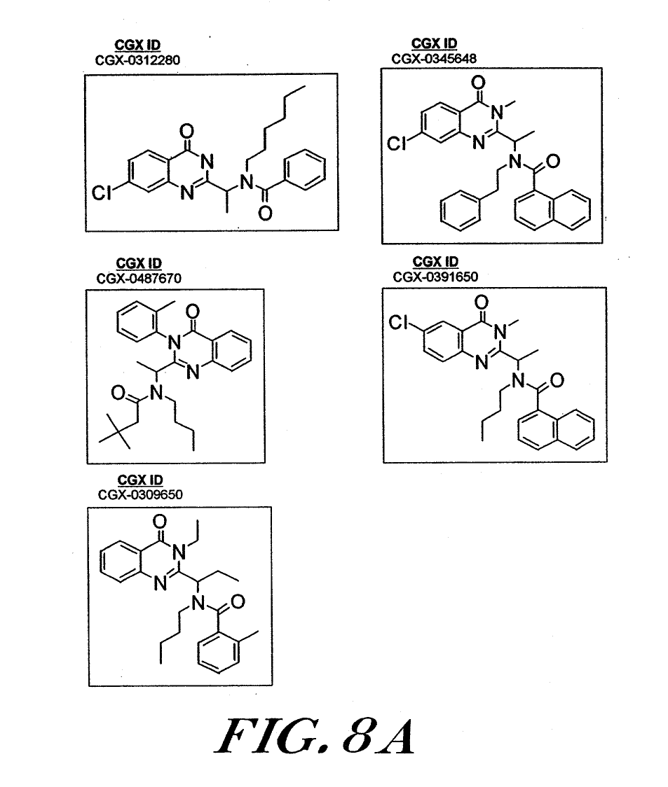

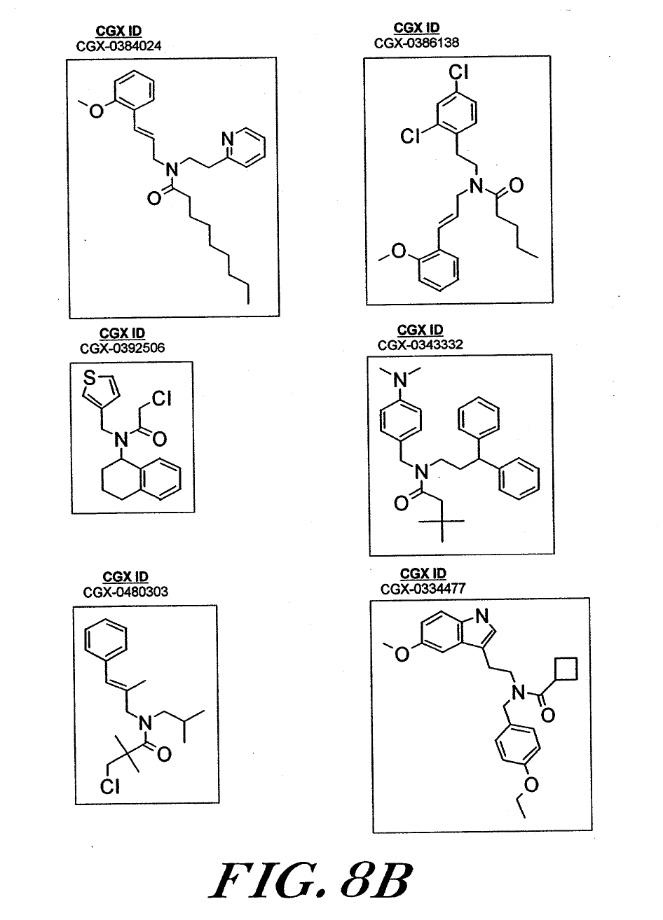

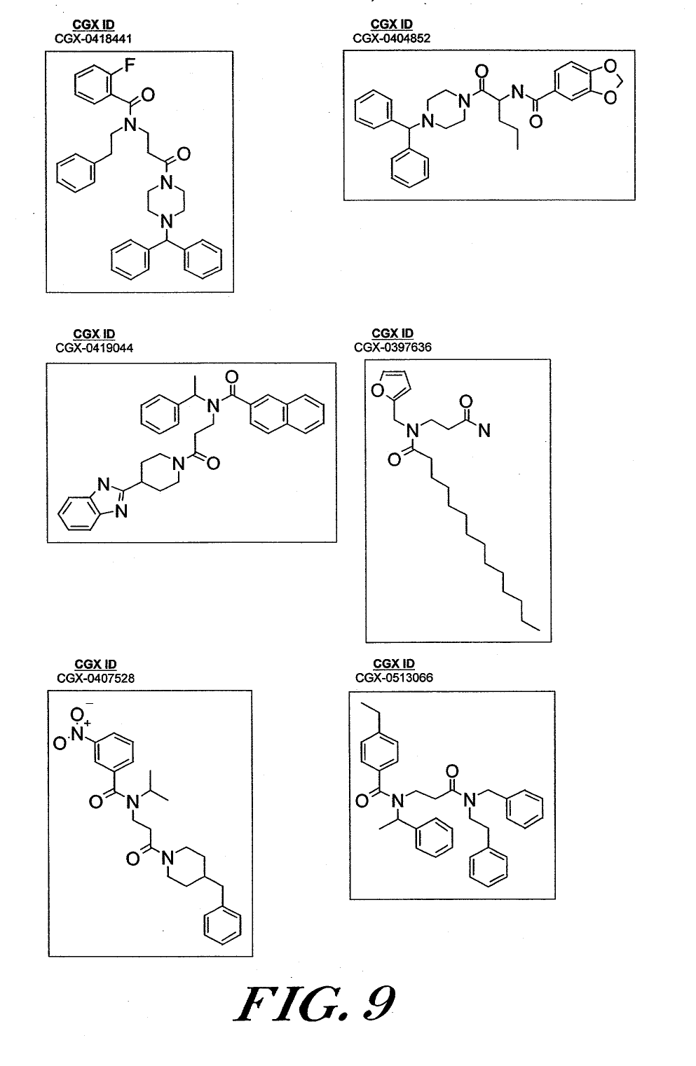

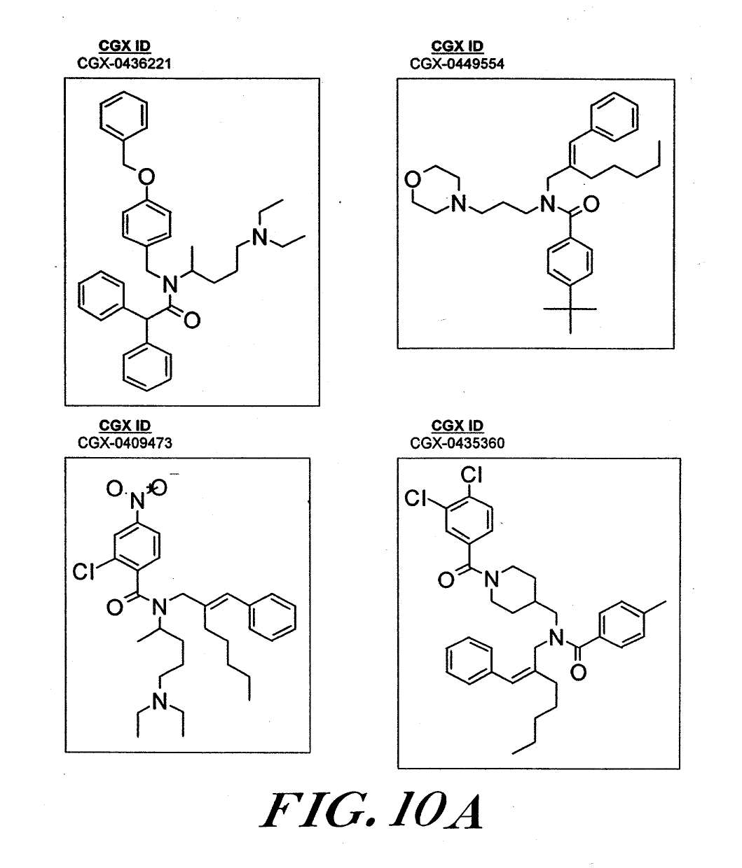

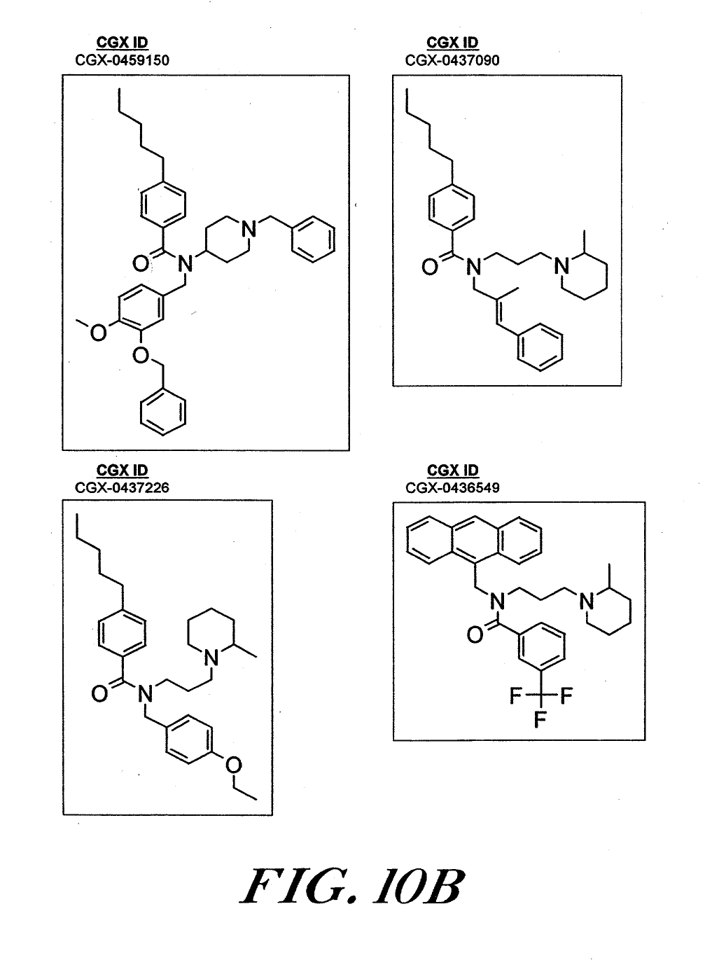

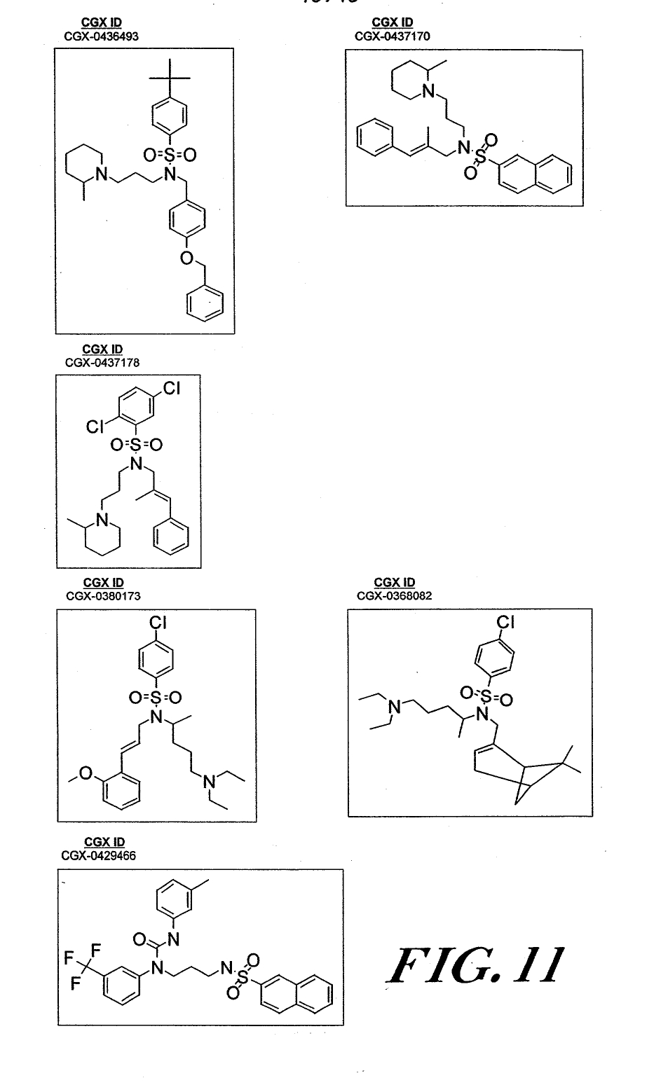

### DETAILED DESCRIPTION OF THE INVENTION

The present invention pertains to compounds effective at modulating fatty acid or triglyceride (“fat”) accumulation by cells, in particular, fat cells which have potential therapeutic applications in the regulation body mass, the treatment of overweight individuals and obesity, and treatment of metabolic disorders.

More particularly, the present invention relates to a method of modulating the accumulation of a fatty acid or triglyceride in a cell, comprising a step of contacting said cell with a compound, wherein said compound comprises a substituted or unsubstituted aryl group and an amide (i.e., N—CO), sulfonamide (i.e., SO₂—N), or ureylene group (i.e., N—CO—N), such that modulation of said fatty acid or triglyceride accumulation occurs.

The present invention features compounds and methods of modulating, i.e., increasing or decreasing, the accumulation (e.g., uptake) of fatty acids or triglycerides by cells, e.g., adipocytes or preadipocytes, in vivo or in vitro, comprising a step of contacting a cell with a amide, sulfonamide, or ureylene-containing compound, such as those according to the following Formula:

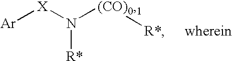

Ar is a substituted or unsubstituted aryl group,

X is L or SO₂,

R* is an organic moiety, and

L is a linking group,

and pharmaceutically acceptable salts thereof.

In other embodiments, the present invention features compounds and methods of modulating, i.e., increasing or decreasing, the accumulation (e.g., uptake) of fatty acids or triglycerides by cells, e.g., adipocytes or preadipocytes, in vivo or in vitro, comprising a step of contacting a cell with a amide, sulfonamide, or ureylene-containing compound, such as those according to the following Formulae:

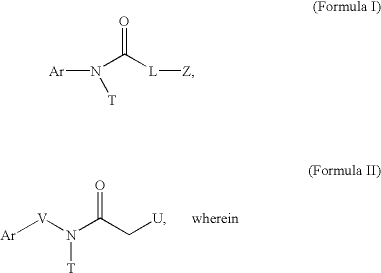

Ar is a substituted or unsubstituted aryl group,

L is a linking group, and

V is a substituted or unsubstituted C₁₋₆ straight or branched chain alkylene group, or a substituted or unsubstituted C₂₋₆ straight or branched chain alkenylene or alkynylene group,

T is a hydrogen or a C₁₋₅ straight or branched chain alkyl group,

U is a halogen, or a C(halogen)₃, CH(halogen)₂, CH₂(halogen), alkyl, or nitro group, and

Z is a substituted alkyleneamine or substituted amine derivative;

said compound having the property of modulating the accumulation of fatty acids or triglycerides by cells;

N and O have their art-recognized meaning, i.e., N meaning nitrogen and O meaning oxygen.

The group L is a “linking group” which is covalently bound to at least two other moieties and may be, for example, a substituted or unsubstituted oligomethylene group. A linking group is a linear chain of carbon atoms which may be optionally substituted or unsaturated. Preferably, a linking group in one embodiment is relatively small compared to the rest of the molecule, and more preferably less than about 250 molecular weight, and even more preferably less than about 75 molecular weight. An especially preferred linking group in some embodiments is an alkylene group of the formula —(CH₂)ₙ— wherein n is 1, 2, or 3, etc. As used herein, L* means a linking group which has a carbonyl at one end, for example —(CH₂)₂(CO)—.

In one embodiment, L is an unsubstituted C₁-C₆ alkylene group. In another embodiment, L is a substituted C₁-C₆ alkylene group.

The linking group L may be, for example, a direct chemical bond, (CR^{a}R^{b})ₙ, CR^{a}OR^{b}(CR^{c}R^{d})ₙ, CR^{a}SH(CR^{c}R^{d})ₙ, CR^{a}NR^{b}R^{c}(CR^{d}R^{e})ₙ, (CR^{a}R^{b})ₙO(CR^{c}R^{d})ₙ, wherein each n is independently either 0, 1, 2, or 3, and R^{a}, R^{b}, R^{c}, R^{d}, and R^{e} are each independently hydrogen, a substituted or unsubstituted C₁-C₅ branched or straight chain alkyl or alkoxy, C₂-C₅ branched or straight chain alkenyl, aryloxycarbonyl, arylaminocarbonyl, arylalkyl, acyl, aryl, or C₃-C₈ ring group.

In some preferred embodiments, L is (CR^{a}R^{b})ₙ wherein n is either 0, 1, 2, or 3, and R^{a} and R^{b} are each independently hydrogen, a substituted or unsubstituted C₁-C₅ branched or straight chain alkyl or alkoxy, arylalkyl, aryl, or a C₃-C₈ cycloalkyl group.

In more preferred embodiments, L is (CR^{a}R^{b})ₙ wherein n is either 0, 1, 2, or 3, and R^{a} and R^{b} are each independently hydrogen, methyl, benzyl, phenylethyl, sec-phenylethyl, iso-butyl, or iso-propyl group.

Some more preferred L groups are (CH₂)₂ and CH(CH₃)(CH₂).

Hydrogen is a preferred T group, and the halogens (i.e., F, Cl, Br, and I) are preferred U groups.

Some preferred V groups include —CH₂(HC═CH)— (both E and Z configurations) and —(CH₂)ₜ— wherein t is 1, 2, 3 or 4.

Some preferred Z groups include the following:

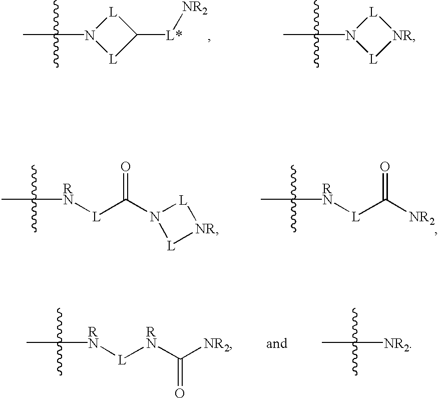

Therefore, the use of the following compounds is also within the scope of the present invention:

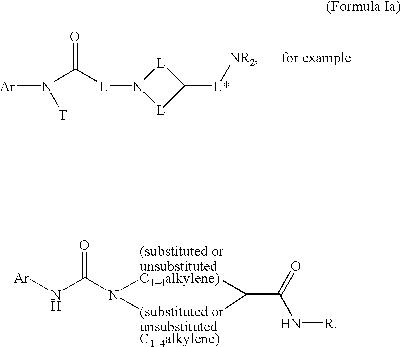

The use of compounds according to the following formula is also within the scope of the present invention:

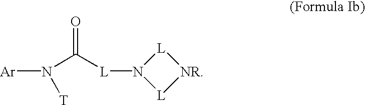

The use of compounds according to the following formula is also within the scope of the present invention:

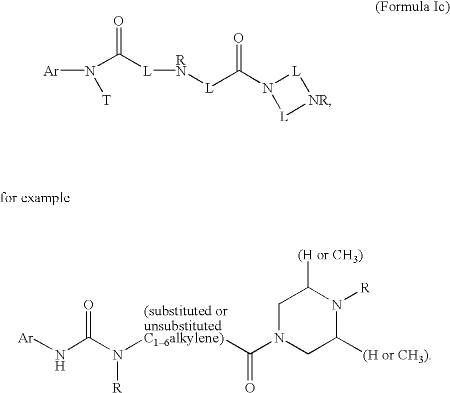

The use of compounds according to the following formula is also within the scope of the present invention:

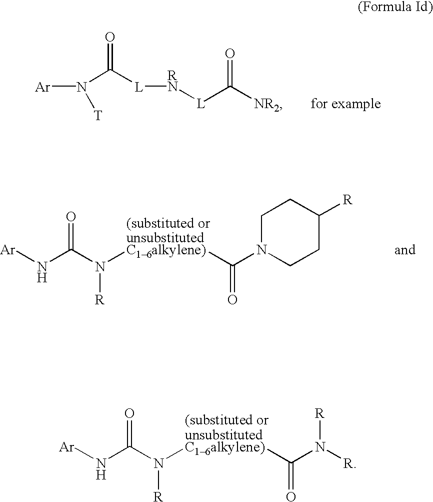

The use of compounds according to the following formula is also within the scope of the present invention:

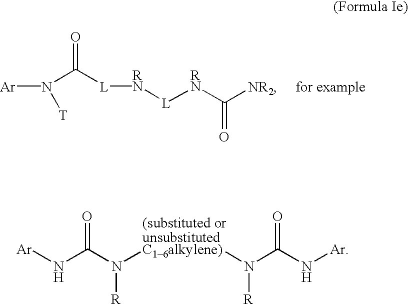

The use of compounds according to the following formula is also within the scope of the present invention:

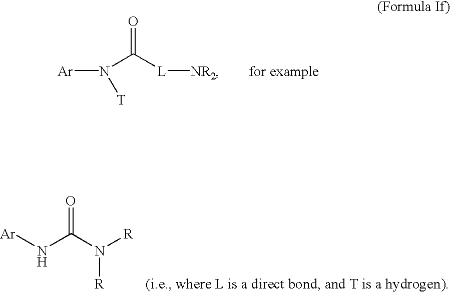

Still further examples of preferred Z groups include the following:

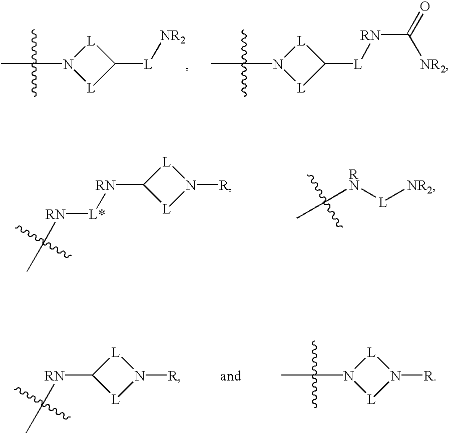

Accordingly, the use of the following compounds is also within the scope of the present invention:

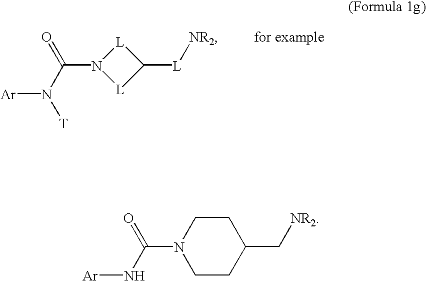

The use of compounds according to the following formula is also within the scope of the present invention:

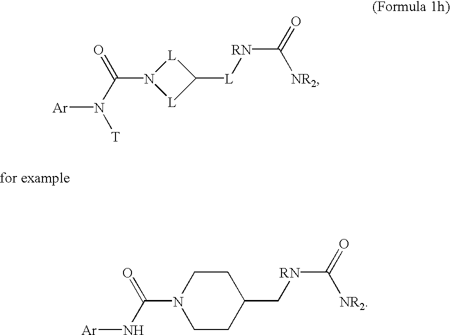

The use of compounds according to the following formula is also within the scope of the present invention:

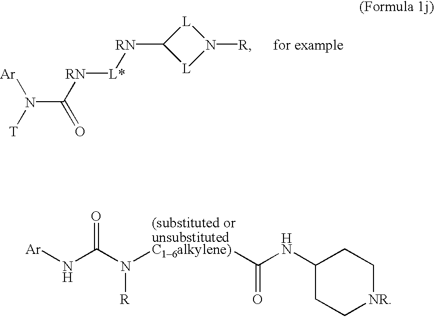

The use of compounds according to the following formula is also within the scope of the present invention:

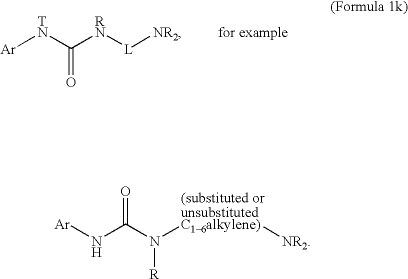

The use of compounds according to the following formula is also within the scope of the present invention:

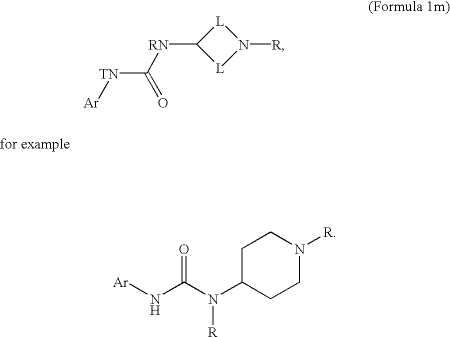

The use of compounds according to the following formula is also within the scope of the present invention:

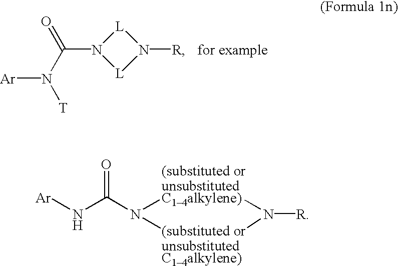

In one preferred embodiment, the use of compounds according to the following formula is also within the scope of the present invention:

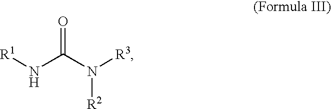

wherein each R group, in this Formula and as used generally throughout the description of the present invention, is independently selected from the group consisting of a hydrogen atom, a substituted or unsubstituted straight or branched alkyl, substituted or unsubstituted cycloalkyl, substituted or unsubstituted alkenyl, substituted or unsubstituted alkynyl, substituted or unsubstituted heterocyclic, substituted or unsubstituted carbocyclic, substituted or unsubstituted aryl, substituted or unsubstituted aralkyl, substituted or unsubstituted aryloxyalkyl, substituted or unsubstituted arylacetamidoyl, substituted or unsubstituted alkylaryl, substituted or unsubstituted heteroaralkyl, substituted or unsubstituted alkylcarbonyl, substituted or unsubstituted arylcarbonyl, substituted or unsubstituted heteroarylcarbonoyl, or substituted or unsubstituted heteroaryl group; or two R groups taken together when bound to the same nitrogen atom form a substituted or unsubstituted heterocyclic ring; and (CR′R″)₁₋₁₂H, (CR′R″)₀₋₃NR′R″, (CR′R″)₀₋₃CN, (CR′R″)₀₋₃NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′; wherein each of R′ and R″ is independently hydrogen, a C₁-C₅ alkyl, C₂-C₅ alkenyl, C₂-C₅ alkynyl, aryl—(C₁-C₅ alkyl), or aryl group, or R′ and R″ taken together are a benzylidene group or a —(CH₂)ₙO(CH₂)ₙ— (wherein each n is 1, 2, or 3) group; and pharmaceutically acceptable salts thereof.

In certain preferred aspects of the invention, the R groups are independently selected from the group consisting of a hydrogen atom, a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted aryl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; or two R groups taken together when bound to the same nitrogen atom form a substituted or unsubstituted morpholine or piperidine ring; and (CR′R″)₀₋₃NH₂, (CR′R″)₀₋₃CN, (CR′R″)₀₋₃NO₂, (CR′R″)₀₋₃CF₃, (CR′R″)₀₋₃CHF₂, (CR′R″)₀₋₃CH₂F, (CR′R″)₀₋₃CONH₂, (CR′R″)₀₋₃S(O)₁₋₂NH₂, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂H, or (CR′R″)₀₋₃OH; wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

In another preferred aspect of the invention, the R groups are independently selected from the group consisting of a hydrogen atom, a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted phenyl or naphthyl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; or two R groups taken together when bound to the same nitrogen atom form a substituted or unsubstituted morpholine or piperidine ring; and (CH₂)₁₋₃NH₂, (CH₂)₁₋₃CN, (CH₂)₁₋₃NO₂, (CH₂)₁₋₃CF₃, (CH₂)₁₋₃CHF₂, (CH₂)₁₋₃CH₂F, (CH₂)₁₋₃CONH₂, (CH₂)₁₋₃S(O)₁₋₂NH₂, (CH₂)₁₋₃CHO, (CH₂)₁₋₃O(CH₂)₁₋₃H, (CH₂)₁₋₃S(O)₀₋₂H, (CH₂)₁₋₃H, (CH₂)₁₋₃COH, (CH₂)₁₋₃CO₂H, or (CH₂)₁₋₃OH.

In one embodiment, the compounds of Formula III have R¹ as a substituted phenyl group.

In other aspects of the invention, the following compounds may be used

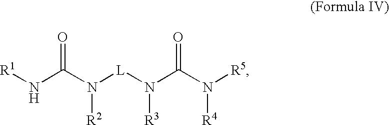

wherein each R group is as defined above, and L is a linking group. In one aspect, compounds according to Formula IV have R¹ as a substituted phenyl group. In another embodiment of the invention, R² of Formula IV is hydrogen. In still another embodiment, R⁴ of Formula IV is hydrogen. Additionally, the invention relates to the use of compounds of Formula IV, wherein R⁵ is a substituted phenyl group, a naphthyl group, or a cycloalkyl group.

The L and R² groups of Formula IV may be taken together to form a cyclic alkylene group according to the following structure

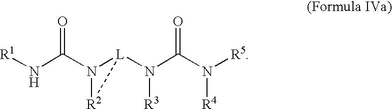

For example, compounds according to the following structure are within the scope of the present invention

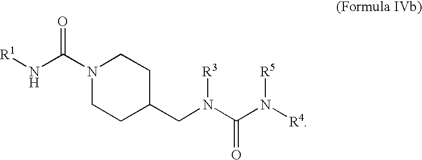

In other aspects, the present invention relates to the use of compounds having the structure

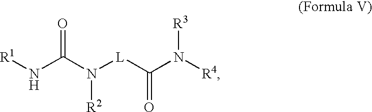

wherein each R group is as defined above, and L is a linking group. In one aspect, R¹ of Formula V is a substituted alkyl group. In another embodiment, R¹ of Formula V is a substituted aralkyl group. In still another embodiment of Formula V, R³ and R⁴ are taken together to form a heterocyclic moiety, for example, the compounds having the following structure

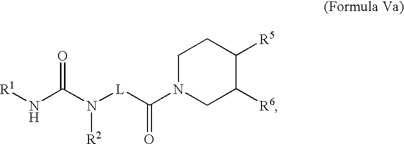

wherein L is a linking group, R⁵ is an R group as defined above, and R⁶ is hydrogen or a an alkyl group. In one embodiment of Formula Va, R¹ is a substituted alkyl group. In another embodiment of Formula Va, R¹ is a substituted aralkyl group. In still another embodiment of Formula Va, R⁶ is a methyl group.

The invention also pertains to the use of compound having the structure

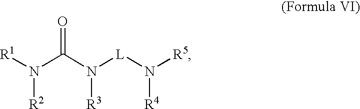

wherein each R group is as defined above, and L is a linking group. In one aspect of Formula VI, R¹ is a substituted phenyl group. In another aspect, compounds according to Formula VI have R² as a hydrogen. In yet another embodiment, L and R³ of Formula VI may be taken together to form a cyclic alkylene group according to the following structure

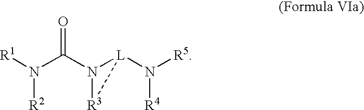

For example, L and R³ of Formula VI may be taken together to form a cyclic alkylene group according to the following structure

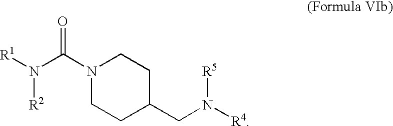

In one embodiment, R¹ in Formula VIb is a substituted phenyl group. In another aspect, R² of Formula VIb is a hydrogen.

The invention likewise pertains to the use of compounds having the following structure

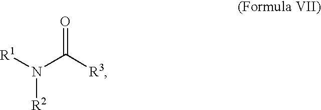

wherein each R group is as defined above. In certain aspects, R¹ and R² of Formula VII are independently a substituted or unsubstituted C₁-C₆ alkyl group or a substituted or unsubstituted C₂-C₆ alkenyl group. In another embodiment, R³ of Formula VII is a substituted or unsubstituted C₁-C₆ alkyl group or a substituted or unsubstituted phenyl or naphthyl group. R¹ and R² of Formula VII may also be taken together to form a heterocyclic moiety, for example as in the compound having the following structure

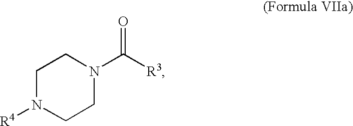

wherein R⁴ is an R group as defined above.

In other aspects of the present invention, the compounds having the following structure are within the invention

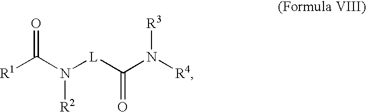

wherein each R group is as defined above, and L is a linking group. R³ and R⁴ in Formula VII may also be taken together to form a heterocyclic moiety, for example, as in the compounds having the structure

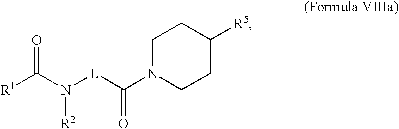

wherein R⁵ is an R group as defined above. Likewise, R³ and R⁴ in Formula VII may also be taken together to form a heterocyclic moiety, for example, as in the compounds having the structure

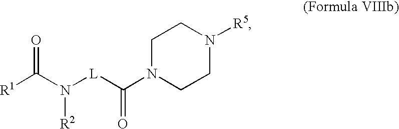

wherein R⁵ is an R group as defined above.

In other embodiments, the use of the compounds according to the following structure is with in the scope of the invention

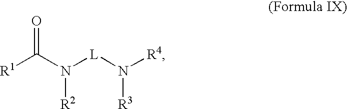

wherein each R group is as defined above, and L is a linking group. In one aspect of Formula IX, R¹ is a substituted phenyl group. In another aspect of Formula IX, R² is a substituted or unsubstituted C₁-C₆ alkyl group. In yet another embodiment of Formula IX, R³ and R⁴ are independently a substituted or unsubstituted C₁-C₆ alkyl group. Additionally, R³ and L of Formula IX may be taken together to form a cyclic alkylene group according to the following structure

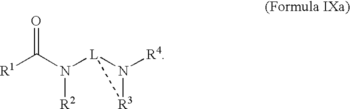

In another embodiment, R² and L of Formula IX may be taken together to form a cyclic alkylene group according to the following structure

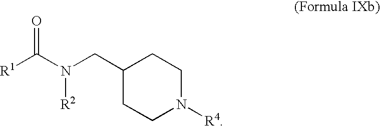

In one aspect of Formula IXa or IXb, R¹ is a substituted phenyl group. In another embodiment of Formula IXa or IXb, R² is a substituted or unsubstituted C₁-C₆ alkyl group. In another embodiment of Formula IXa or IXb, R³ and R⁴ are taken together to form a heterocyclic moiety, for example, the compounds having the structure

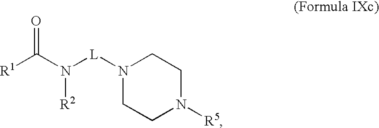

wherein R⁵ is an R group as defined above.

Similarly, the invention also relates to the use of compounds having the structure

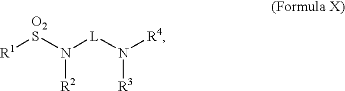

wherein each R group is as defined in above, and L is a linking group. In one aspect of Formula X, R¹ is a substituted phenyl group or a naphthyl group. In another embodiment of Formula X, R² is a substituted or unsubstituted C₁-C₁₂ alkyl group or a substituted or unsubstituted C₂-C₁₂ alkenyl group. In yet another embodiment of Formula X, R³ and R⁴ are independently a substituted or unsubstituted C₁-C₁₂ alkyl group or a substituted or unsubstituted C₂-C₁₂ alkenyl group. In still another embodiment of Formula X, R³ and R⁴ are taken together to form a heterocyclic moiety, for example, the compounds having the following structure

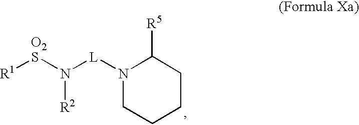

wherein R⁵ is hydrogen or a an alkyl group (e.g., a methyl group). In one embodiment of Formula Xa, R¹ is a substituted phenyl group or a naphthyl group. In another embodiment of Formula Xa, R² is a substituted or unsubstituted C₁-C₁₂ alkyl group or a substituted or unsubstituted C₂-C₁₂ alkenyl group.

In the compounds of the invention, “Ar” is an aryl group.

In general, the term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. “Aryl” therefore includes both heteroaromatic and non-heteroaromatic moieties, unless otherwise indicated.

Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls,” or “heteroaromatics”. Aryl groups may also be fused or bridged with alicyclic or heterocyclic rings which are not aromatic so as to form a polycycle (e.g., tetralin).

The aromatic ring may be substituted at one or more ring positions with such substituents as described above, as for example, halogen, hydroxyl, alkyl (e.g., tolyl), alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. An aryl group may also be substituted with an X group, defined elsewhere herein.

Preferred Ar substituents according to the Formulae of the invention include a substituted or unsubstituted naphthyl group or

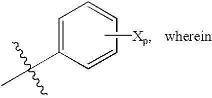

p is an integer from zero to five inclusive (preferably p is one or two),

X is selected from the group consisting of a substituted or unsubstituted straight or branched alkyl, substituted or unsubstituted cycloalkyl, substituted or unsubstituted alkenyl, substituted or unsubstituted alkynyl, substituted or unsubstituted heterocyclic, substituted or unsubstituted carbocyclic, substituted or unsubstituted aryl, substituted or unsubstituted aralkyl, substituted or unsubstituted aryloxyalkyl, substituted or unsubstituted arylacetamidoyl, substituted or unsubstituted alkylaryl, substituted or unsubstituted heteroaralkyl, substituted or unsubstituted alkylearbonyl, substituted or unsubstituted arylcarbonyl, substituted or unsubstituted heteroarylcarbonoyl, or substituted or unsubstituted heteroaryl group; and (CR′R″)₁₋₁₂H, (CR′R″)₀₋₃NR′R″, (CR′R″)₀₋₃CN, (CR′R″)₀₋₃NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′; wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

In certain preferred aspects of the invention, X is independently selected from the group consisting of a substituted or unsubstituted straight or branched C₁-C₁₀ alkyl, substituted or unsubstituted C₃-C₈ cycloalkyl, substituted or unsubstituted C₂-C₁₀alkenyl, substituted or unsubstituted C₂-C₁₀ alkynyl, substituted or unsubstituted heterocyclic, substituted or unsubstituted carbocyclic, substituted or unsubstituted aryl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; and (CR′R″)₀₋₃NH₂, (CR′R″)₀₋₃CN, (CR′R″)₀₋₃NO₂, (CR′R″)₀₋₃CF₃, (CR′R″)₀₋₃CHF₂, (CR′R″)₀₋₃CH₂F, (CR′R″)₀₋₃CONH₂, (CR′R″)₀₋₃S(O)₁₋₂NH₂, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂H, or (CR′R″)₀₋₃OH; wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

In another aspect of the invention, X is selected from the group consisting of a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted phenyl or naphthyl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; and (CH₂)₁₋₃NH₂, (CH₂)₁₋₃CN, (CH₂)₁₋₃NO₂, (CH₂)₁₋₃CF₃, (CH₂)₁₋₃CHF₂, (CH₂)₁₋₃CH₂F, (CH₂)₁₋₃CONH₂, (CH₂)₁₋₃S(O)₁₋₂NH₂, (CH₂)₁₋₃CHO, (CH₂)₁₋₃₀O(CH₂)₁₋₃H, (CH₂)₁₋₃S(O)₀₋₂H, (CH₂)₁₋₃O(CH₂)₁₋₃H, (CH₂)₁₋₃COH, (CH₂)₁₋₃CO₂H, or (CH₂)₁₋₃OH.

In one embodiment, X is a halogen, C(halogen)₃, CH(halogen)₂, CH₂(halogen), alkyl, or nitro group.

In another embodiment, X is CF₃, CCl₃, CHF₂, CHCl₂, F, Cl, Br, I, NO₂, C₂-C₁₀ n-alkyl group, CN, OCH₃, CH₃, phenoxy, phenyl, OCH₂CH₃, or CH₂CH₃.

In yet another embodiment X is a methyl, ethyl, propyl, butyl, pentyl, hexyl, heptyl, octyl, or nonyl group.

In still yet another embodiment, p is 1, and X is o-F, o-Me, p-OCH₃, m-F, m-CN, m-CF₃, m-Cl, p-NO₂, p-phenoxy, m-CH₃, p-Cl, p-Br, o-phenyl, p-CF₃, p-ethyl, p-ethoxy, m-Br, or m-NO₂.

Additionally, in another aspect, p is 2, and both X groups are m,m-F₂, m,p-O₂(CH₂), m,p-Cl₂, o,o-(CH₃)₂, or o,p-Cl₂.

By way of example, the following Ar groups in the Formulae within the scope of the present invention:

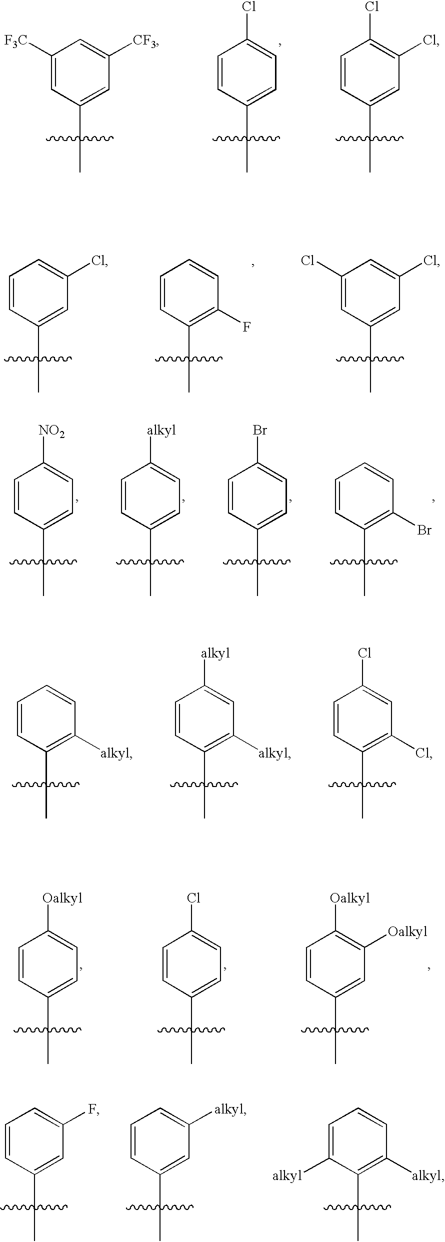

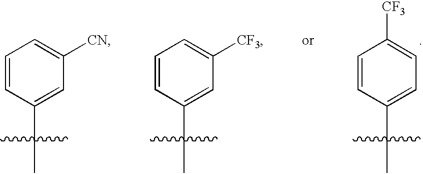

Among these Ar groups, the alkyl group is preferably an n-alkyl (especially n-heptyl) or iso-alkyl (especially iso-propyl) group.

A most preferred Ar group is

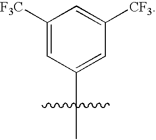

The R group of any Formula herein may be a substituted or unsubstituted branched, bicyclic, cyclic, or unbranched C₁-C₂₀ alkyl group or a substituted or unsubstituted branched, bicyclic, cyclic, or unbranched C₂-C₂₀ alkylene group.

In another embodiment, the R group of any Formula herein may be an iso-propyl, methyl, iso-butyl, 2-benzylideneheptyl, sec-butyl, cyclohexyl, cyclopropyl, 2-(N-morpholinyl)-ethyl, a sec-phenylethyl or phenylethyl group.

When the R group is an alkyl group, the following are preferred: an iso-propyl, methyl, iso-butyl, sec-butyl, heptyl (including substituted versions there of, e.g., 2-benzylideneheptyl), ethyl (including substituted versions there of, e.g., 2-(N-morpholinyl)-ethyl), or butyl group is preferred. Among cycloalkyl R groups, cyclohexyl and cyclopropyl groups are preferred. Among aralkyl R groups, sec-phenylethyl and phenylethyl groups are preferred.

In another aspect of the invention, the R group of any Formula herein may be a cyclopropyl or cyclohexyl group.

The R group may also be any of the following carbonyl derivatives:

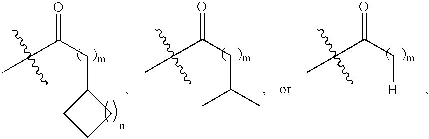

wherein each m is an integer from 1 to 8 inclusive, and each n is an integer from 0 to 5 inclusive;

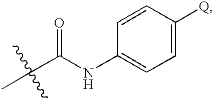

wherein Q is a halogen, hydrogen, or a C₁-C₅ alkyl, O(C₁-C₅ alkyl), or benzyloxy group;

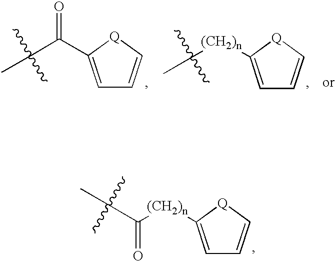

wherein Q is NH, O, or S, and n is 1, 2, or 3; or a benzoyl group, including the following substituted versions thereof:

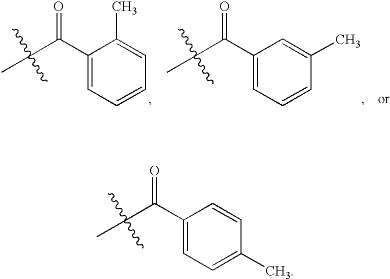

The R group may also be

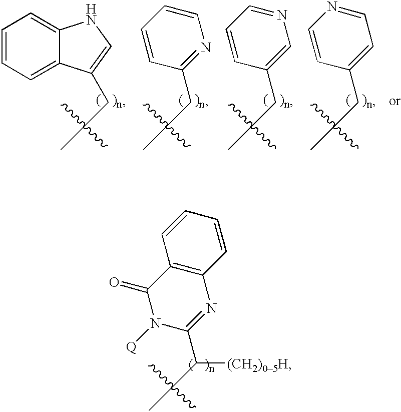

wherein n is an integer from 1 to 3 inclusive, and Q is a C₁-C₅ alkyl, C₂-C₅ alkenyl, or C₂-C₅ alkynyl group; or

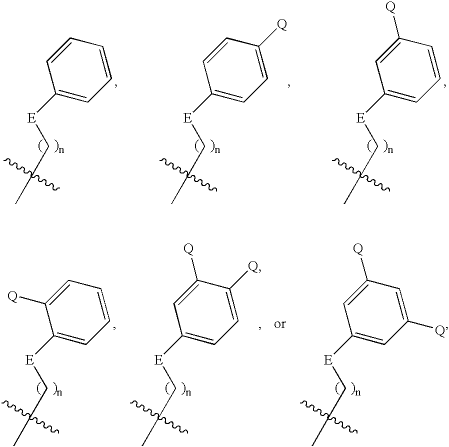

wherein n is an integer from 0 to 5 inclusive, and Q is a C₁-C₅ alkyl, O(C₁-C₅ alkyl), or benzyloxy group, or Q is a halogen, and E is a direct bond or an oxygen atom.

Additionally, two R groups may be taken together when bound to the same nitrogen atom to form a piperidine ring thus:

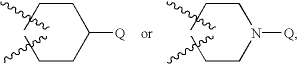

wherein Q is an R group as defined above, or preferably a substituted or unsubstituted alkyl, substituted or unsubstituted aralkyl, or substituted or unsubstituted heteroaryl group, or hydrogen. Another preferred Q group is a benzyl group or

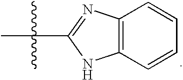

Still further preferred R groups according to the invention are the following:

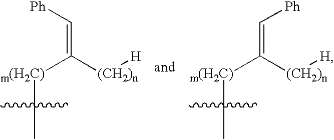

where m is 0, 1, 2, or 3, and n is 1, 2, 3, 4, 5, 6, or 7.

Also, an R group may be a (CR′R″)₀₋₃CH(phenyl)₂ group; wherein R′ and R″ are each independently hydrogen, a C₁-C₅ alkyl, C₂-C₅ alkenyl, C₂-C₅ alkynyl.

Likewise, an R group may also be a naphthyl group, or a partially hydrogenated derivative thereof. Similarly, and R group may be any of the following:

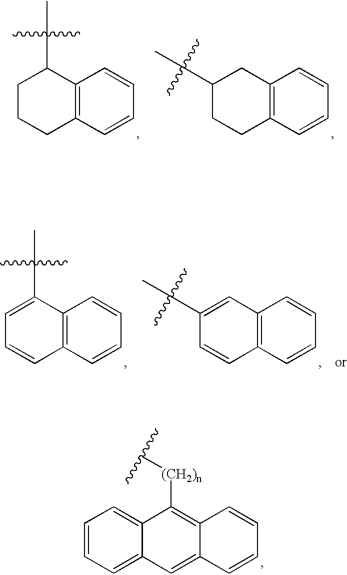

wherein n is an integer from 1 to 4.

Additionally, and R group may be

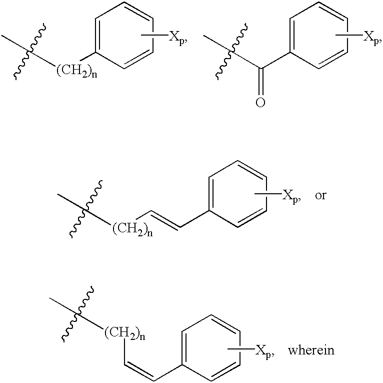

n is an integer from zero to six inclusive,

p is an integer from zero to five inclusive,

X is selected from the group consisting of a substituted or unsubstituted straight or branched alkyl, substituted or unsubstituted cycloalkyl, substituted or unsubstituted alkenyl, substituted or unsubstituted alkynyl, substituted or unsubstituted heterocyclic, substituted or unsubstituted carbocyclic, substituted or unsubstituted aryl, substituted or unsubstituted aralkyl, substituted or unsubstituted aryloxyalkyl, substituted or unsubstituted arylacetamidoyl, substituted or unsubstituted alkylaryl, substituted or unsubstituted heteroaralkyl, substituted or unsubstituted alkylcarbonyl, substituted or unsubstituted arylcarbonyl, substituted or unsubstituted heteroarylcarbonoyl, or substituted or unsubstituted heteroaryl group; and (CR′R″)₁₋₁₂H, (CR′R″)₀₋₃NR′R″, (CR′R″)₀₋₃CN, (CR′R″)₀₋₃NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃H, (CR′R″)₀₋₃COR′, (CR′R″)₀₋₃CO₂R′, or (CR′R″)₀₋₃OR′; wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

An R group may also be any of the following:

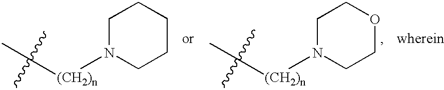

n is an integer from zero to six inclusive.

The present invention also relates to a method of modulating the accumulation of a fatty acid or triglyceride, wherein the compound is selected from those depicted in the accompanying Drawings, and pharmaceutically acceptable salts thereof.

In preferred aspects, the invention relates to a method of modulating the accumulation of a fatty acid or triglyceride, wherein the compound is selected compound numbers AGX-0034, AGX-0020, AGX-0088, AGX-0018, AGX-0042, AGX-0099, AGX-0013, AGX-0025, and AGX-0008 in Table 1 below and the accompanying drawings.

The term “substituted” includes substituents which may be placed on the moiety and which allow the molecule to perform its intended function. Examples of substituents include straight and branched chain alkyl (including polycycloalkyl, e.g., bicycloalkyl), alkenyl, alkynyl, aryl (including heteroaryl and the “Ar” groups defined above), (CR′R″)₀₋₃NR′R″(including NH₂ and dialkylamino), (CR′R″)₀₋₃CN (including CN), (CR′R″)₀₋₃NO₂ (including NO₂), halogen (e.g., F, Cl, Br, I), (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′ (including CO₂H), or (CR′R″)₀₋₃OR′ groups; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)ₙO(CH₂)ₙ— (where each n is 1, 2, or 3) group. Preferably, substitutions enhance the ability of the compounds of the invention modulating compound to perform its intended function, e.g., modulate fatty acid or triglyceride accumulation activity.

The term “heterocyclic” includes heteroaryls as well as any ring formed which incorporate a heteroatom or an atom which is not carbon. The ring may be saturated or unsaturated and may contain one or more double bonds. Examples of preferred heterocyclic groups include pyridyl, furanyl, thiophenyl, morpholinyl, and indolyl groups.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The terms C₁-C₆ and C₁₋₆ include alkyl groups containing 1, 2, 3, 4, 5, or 6 carbon atoms.

Moreover, unless otherwise specified, the term alkyl may include both “unsubstituted alkyls” and “substituted alkyls,” the latter of which refers to alkyl moieties having substituents replacing a hydrogen on one or more carbons of the hydrocarbon backbone. Such substituents may include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls may also be further substituted, e.g., with the substituents described above. An “alkylaryl” or an “arylalkyl” moiety is an alkyl substituted with an aryl (e.g., phenylmethyl (i.e., benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The terms C₂-C₆ and C₂₋₆ include alkenyl groups containing 2, 3, 4, 5, or 6 carbon atoms.

Moreover, unless otherwise specified, the term alkenyl may include both “unsubstituted alkenyls” and “substituted alkenyls,” the latter of which refers to alkenyl moieties having substituents replacing a hydrogen on one or more carbons of the hydrocarbon backbone. Such substituents may include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The terms C₂-C₆ C₂₋₆ include alkynyl groups containing 2, 3, 4, 5, or 6 carbon atoms.

Moreover, unless otherwise specified, the term alkynyl may include both “unsubstituted alkynyls” and “substituted alkynyls,” the latter of which refers to alkynyl moieties having substituents replacing a hydrogen on one or more carbons of the hydrocarbon backbone. Such substituents may include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. The term “substituted acyl” includes acyl groups where one or more of the hydrogen atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthylcarboxy, etc.

The terms “alkoxyalkyl,” “alkylaminoalkyl,” and “thioalkoxyalkyl” include alkyl groups, as described above, which further include oxygen, nitrogen, or sulfur atoms, respectively, replacing one or more carbons of the hydrocarbon backbone, e.g., oxygen, nitrogen, or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups may be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl,” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide” or “aminocarboxy” includes compounds or moieties which contain a nitrogen atom which is bound to the carbon of a carbonyl or a thiocarbonyl group. The term includes “alkaminocarboxy” groups which include alkyl, alkenyl, or alkynyl groups bound to an amino group bound to a carboxy group. It includes arylaminocarboxy groups which include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic” refer to two or more cyclic rings (e.g., cycloalkyls, cycloalkenyls, cycloalkynyls, aryls or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, suifliydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur, and phosphorus.

It will be noted that the structures of some of the compounds of this invention include stereogenic carbon atoms. It is understood accordingly that the isomers arising from such asymmetry (e.g., all enantiomers and diastereomers) are included within the scope of this invention, unless indicated otherwise. Such isomers may be obtained in substantially pure form by classical separation techniques and by stereochemically controlled synthesis. Furthermore, the structures and other compounds and moieties discussed in this application also include all tautomers thereof.

The compounds of the invention also include prodrugs. Prodrugs of the invention may or may not be able to interact with a biological target prior to being metabolized in vivo. However, once the compounds of the invention which are prodrugs are metabolized in vivo or in vitro, they are capable of performing their intended function, e.g., modulate fatty acid or triglyceride accumulation.

The present invention therefore also relates to pharmaceutical compositions for use in the methods described herein. Similarly, the present invention relates to a prodrug pharmaceutical composition for use in the methods described herein.

In one embodiment, a prodrug compound of the invention is capable of performing the intended function after being orally administered. In order to perform the intended function after oral administration, it is believed that a compound must be absorbed by a portion of the digestive tract. In one embodiment of the invention, a prodrug compound of the invention is capable of being absorbed by the digestive tract.

The present invention is also related to prodrugs. Prodrugs are compounds which are converted in vivo to active forms (see, e.g., R. B. Silverman, 1992, “The Organic Chemistry of Drug Design and Drug Action”, Academic Press, Chp. 8). Prodrugs may be used to alter the biodistribution (e.g., to allow compounds which would not typically enter the reactive site of the protease) or the pharmacokinetics for a particular compound. For example, a carboxylic acid group, may be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group may be esterified with moieties (e.g., acyloxymethyl esters) which are cleaved to reveal an intermediate compound which subsequently decomposes to yield the active compound. The prodrug moieties may be metabolized in vivo by esterases or by other mechanisms to carboxylic acids.

Examples of prodrugs and their uses are well known in the art (see, e.g., Berge et al. (1977) “Pharmaceutical Salts”, J. Pharm. Sci. 66:1-19). The prodrugs may be prepared in situ during the final isolation and purification of the compounds, or by separately reacting the purified compound in its free acid form with a suitable derivatizing agent. Carboxylic acids may be converted into esters via treatment with an alcohol in the presence of a catalyst. Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

Therapeutic Compounds and Uses

The invention provides methods of modulating fatty acid or triglyceride accumulation (e.g., uptake) that feature contacting a cell with a fatty acid or triglyceride modulator (or derivative thereof) of any Formula herein, with preferred modulators having the structures set forth in Table I herein and the accompanying Drawings, such that fatty acid or triglyceride accumulation by the cells is achieved.

The phrase “contacting a cell” includes contacting a cell either in vitro or in vivo. Contacting cells in vivo includes administering a compound (or composition comprising said compound) to a subject such that said compound in such a manner that the compound comes into proximity with the intended target cells, allowing the compound to perform its intended function.

The present invention also features methods of modulating fatty acid or triglyceride accumulation that feature administering to a subject in need thereof, a fatty acid or triglyceride modulator (or derivative thereof) of any Formula herein, with preferred modulators having the structures set forth in Table I herein and the accompanying Drawings, said compound having the property of modulating the accumulation of fatty acids or triglycerides by cells.

In one aspect, the invention relates to a method of modulating the accumulation of a fatty acid or triglyceride in a cell, comprising a step of contacting said cell with a compound, wherein said compound comprises a substituted or unsubstituted aryl group and an amide, sulfonamide, or ureylene group, such that modulation of said fatty acid or triglyceride accumulation occurs. The modulating property is an increase in the accumulation of fatty acids or triglycerides by cells, or the modulating property is a decrease in the accumulation of fatty acids or triglycerides by cells. The modulation of said fatty acid or triglyceride uptake is a means of treating or preventing a disease or condition in a subject, particularly where the subject is affected with such a disease or condition, has a susceptibility thereto, or has a medical history thereof. Among the diseases and conditions which may be treated are body weight disorders, cancer, AIDS, diabetes, coronary disease, lipodystrophy, hypertension, cachexia, anorexia nervosa, bulemia nervosa, hyperinsulinemia, stroke, congestive heart failure, gall stones, gout, hyperlipiedemia, hypercholesterolemia, atherosclerosis or arteriosclerosis, metabolic syndrome; a susceptibility thereto, a medical history thereof, or a pathological consequence thereof.

The term “body weight disorder” includes disorders or states associated with growth or metabolism of fat tissue including, but not limited to, rapid weight loss or weight gain, obesity, anorexia, cachexia, bulimia, diabetes, generalized or familial partial lipodystrophy (peripheral fat wasting), hypercholesterolemia, hyperlipidemia, and other diseases of aberrant metabolic rate. A symptom of a body weight disorder is an abnormal body weight which can be determined according to the body mass index (BMI), which is the ratio of [body weight in kg] divided by [height in m]². As used herein, an individual's body weight is defined as being underweight (BMI<18.5), normal (BMI=18.5-24.9), overweight (preobese; BMI=25.0-29.9), moderately overweight (grade 1 obesity; BMI=30.0-34.9), severely overweight (grade 2 obesity; BMI=35.0-39.9), or massively or morbidly obese (grade 3 obesity; BMI=≧40). Ranges intermediate to the above-recited values, e.g., 18.5-24.9, 25.0-29.9, 30.0-34.9, 35.0-39.9, and ≧40, and to the below recited values are also intended to be encompassed by the invention. Body weight disorders also include abnormal or undesirable percentages of body fat. In one embodiment, the percent body fat of said subject is 5% or less, 8% or less, 10% or less, 15% or less, 5% or greater, 10% or greater, 12.5% or greater, 15% or greater, 17.5% or greater, 20% or greater, 25% or greater, 30% or greater, 35% or greater, 40% or greater, etc.

The invention also pertains to a method of treating chronic heart failure in a subject. The invention includes administering to the subject an effective amount of a compound of the invention, e.g., a compound of any one of the Formulae herein.

The invention also pertains to a method of treating left ventricular hypertrophy in a subject. The method includes administering to the subject an effective amount of a compound of the invention, e.g., a compound of any one of the Formulae herein.

Likewise, the invention also pertains to a method of treating acute heart failure in a subject. The method includes administering to the subject an effective amount of a compound of the invention, e.g., a compound of any one of the Formulae herein, such that said acute heart failure in the subject is treated.

The invention also pertains to a method of treating cardiomyopathy in a subject. The method includes administering to the subject an effective amount of a compound of the invention, e.g., a compound of any one of any Formula herein, such that the cardiomyopathy in the subject is treated.

The invention also pertains to a method of treating congestive heart failure in a subject. The method involves administering to the subject a compound of the invention, e.g., a compound of any one of the Formulae herein, such that the congestive heart failure in the subject is treated.

Similarly, the invention also pertains to a method of treating arterial hypertension in a subject. The method includes administering to the subject, an effective amount of a compound of the invention, e.g., a compound of any one of the herein, such that the arterial hypertension in the subject is treated.

The invention also pertains to a method of treating myocardial infarction in a subject. The method includes administering to the subject an effective amount of a compound of the invention, e.g., a compound of any one of the Formulae herein, such that myocardial infarction in the subject is treated.

The invention also pertains to a method for treating vascular stenosis in a subject. The method includes administering to a subject an effective amount of a compound of the invention, e.g., a compound of any one of the Formulae herein, such that the vascular stenosis in the subject is treated.

The invention also pertains to a method for treating a subject for a stroke. The method includes administering to the subject an effective amount of a compound of the invention, e.g., a compound of any one of the Formulae herein, such that the subject is treated for the stroke.

The invention also pertains to a method for treating heart disease in a subject. The method includes administering to the subject an effective amount of a compound of the invention, e.g., a compound of any one of the Formulae herein, such that the subject is treated for heart disease.

In yet another embodiment, the invention pertains to a method for treating diabetes or “metabolic syndrome” (see, Zimmet, “Global and societal implications of the diabetes epidemic” Nature, v.414, p.782, 2001) in a subject. The method includes administering to the subject an effective amount of a fatty acid or triglyceride accumulation modulating compound.

In another further embodiment, the invention also includes a method for treating a state associated with lipid metabolism in a subject. The method includes administering to the subject an effective amount of a fatty acid or triglyceride accumulation modulating compound, such that the state is treated.

The term “state associated with lipid metabolism” includes disorders and states which are caused or modulated (e.g., increased) by abberant, normal, or undesirable (elevated or depressed) levels of lipid metabolism. In certain embodiments, states associated lipid metabolism include, for example, obesity, lipidosis, a lipodystrophy, e.g., hyperlipenia, hyperlipidemia, hyperproteinemia, hyperliposis, lipoidosis, and lipolipoidosis.

In another embodiment, the invention also pertains to a method for treating atherosclerosis in a subject. The method includes administering to the subject an effective amount of a fatty acid or triglyceride accumulation modulating compound.

The terms “treatment,” “treating,” or “treat,” includes the application or administration of a therapeutic agent (e.g., fatty acid or triglyceride modulating compounds) to a subject, or application or administration of a therapeutic agent to an isolated tissue or cell line from a subject, who has a disease or disorder (e.g., a state associated with lipid metabolism) or a symptom of a disease or disorder, such that the disease or disorder (or at least one symptom of the disease or disorder) is cured, healed, prevented, alleviated, relieved, altered, remedied, ameliorated, improved or otherwise affected, preferably in an advantageous manner.

In an embodiment, the invention includes methods and compositions for modifying body weight or the percentage of body fat and treating body weight disorders, including but not limited to, obesity, cachexia, diabetes (particularly Type II diabetes), and anorexia, by administering to the subject an effective amount of fatty acid or triglyceride accumulation modulating compound, such that the body weight disorder is treated or prevented in the subject. An approach which may be used to ameliorate body weight disorders is the administration of fatty acid or triglyceride accumulation modulating compounds, such as those compounds of any one of the Formulae herein.

In an embodiment of the invention, fatty acid or triglyceride accumulation stimulators can be used therapeutically to promote weight gain or increase the percentage of body fat in subjects with an underweight phenotype, e.g., anorexia or cachexia.

Alternatively, symptoms of certain body weight disorders such as, for example, obesity, overweight, and diabetes, which involve an overweight (e.g., a BMI=25.0-29.9 kg/m²) or obese (e.g., a BMI=30.0-34.9, 35.0-39.9, or ≧40 kg/m²) phenotype, can be ameliorated by decreasing the level of fatty acid or triglyceride accumulation with one of the compounds of the invention.

In an embodiment of the invention, inhibitors of fatty acid or triglyceride accumulation can be used therapeutically to reduce weight gain, enhance weight loss or decrease the percentage of body fat in subjects with an overweight or obese phenotype.

The term “administering” includes routes of administration which allow the modulating, e.g., inhibiting, compound to perform its intended function. Examples of routes of administration which may be used include parental injection (e.g., subcutaneous, intravenous, and intramuscular), intraperitoneal injection, oral, inhalation, and transdermal. The injection may be bolus injections or may be continuous infusion. Depending on the route of administration, the fatty acid or triglyceride accumulation modulating, e.g., inhibiting, compound may be coated with or disposed in a selected material to protect it from natural conditions which may detrimentally effect its ability to perform its intended function.

The fatty acid or triglyceride accumulation modulating, e.g., inhibiting, compound may be administered alone or with a pharmaceutically acceptable carrier.

The fatty acid or triglyceride accumulation modulating, e.g., inhibiting, compound also may be administered as a prodrug which is converted to another form in vivo.

The phrase “subject in need” or “subject” includes any subject, e.g., human subject, having a disease or condition that would benefit from direct or indirect modulation of fatty acid or triglyceride accumulation by the cells of said subject, in particular, fatty acid or triglyceride accumulation by the fat cells (e.g., adipocytes or preadipocytes). In one embodiment, a subject is an overweight subject, e.g., an overweight human. In another embodiment, a subject is an obese subject, e.g., an obese human. Such subjects would benefit from administration of inhibitory compounds of the invention.

In another embodiment, a subject is an underweight subject, e.g., an underweight human. Such subjects would benefit from administration of stimulatory compounds of the invention. Overweight, obese, or underweight subjects may include those having a metabolic disorders, e.g., subjects having diabetes or cachexia. Underweight subjects also include, for example, subjects having immune disorders, for example, AIDS patients exhibiting significant or dramatic weight loss.

A subject may also be a companion animal (domesticated or household cats, dogs, etc.). In such cases, the methods of the invention may be applied to under- or overweight companion animals in analogous manner as humans.

A subject may also be a farm animal, and therefor the methods of the present invention apply to animal husbandry. For example, fatty acid or triglyceride accumulation modulating compounds may included in the diet of farm animals (e.g., pigs, cows, lamb/sheep, horses, etc.) in order to produce leaner or fatter livestock.

In a further embodiment, the subject is normal weight, under weight, or over weight subjects as well as transgenic subjects. In one embodiment, the subject has a BMI of 18 or less, 18 or greater, 19 or greater, 20 or greater, 21 or greater, 22 or greater, 23 or greater, 24 or greater, 25 or greater, 26 or greater, 27 or greater, 28 or greater, 29 or greater, 30 or greater, 31 or greater, 32 or greater, 33 or greater, 34 or greater, 35 or greater, 36 or greater, 37 or greater, 38 or greater, 39 or greater, 40 or greater, 41 or greater, 42 or greater, 43 or greater, 44 or greater, or 45 or greater.

The language “therapeutically effective amount” is that amount necessary or sufficient to produce the desired physiologic response, e.g. prevent weight loss or wasting, or treat overweight individuals or obesity, or in the alternative, to prevent or treat secondary effects, e.g., mortality, hypertension, Type 2 diabetes, cardiovascular disease or morbidity, respiratory problems, or cancer. The effective amount may vary depending on such factors as the size and weight of the subject, or the particular fatty acid or triglyceride accumulation modulating, e.g., inhibiting, compound. For example, the choice of the fatty acid or triglyceride accumulation modulating, e.g., inhibiting, compound may affect what constitutes an “effective amount.” One of ordinary skill in the art would be able to study the aforementioned factors and make the determination regarding the effective amount of the fatty acid or triglyceride accumulation modulating, e.g., inhibiting, compound without undue experimentation.

The effective amount may be determined through consideration of the toxicity and therapeutic efficacy of the fatty acid or triglyceride accumulation modulating, e.g., inhibiting, compounds by standard pharmaceutical procedures in cell cultures or experimental animals, e.g., for determining the LD₅₀ (The Dose Lethal To 50% Of The Population) and the ED₅₀ (the dose therapeutically effective in 50% of the population). The dose ratio between toxic and therapeutic effects is the therapeutic index and it may be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to unaffected cells and, thereby, reduce side effects.

The invention also relates to a pharmaceutical composition containing a pharmaceutically acceptable carrier and an effective amount of fatty acid or triglyceride accumulation modulating, e.g., inhibiting, compound. The invention pertains to pharmaceutical compositions comprising a compound of any one of the Formulae herein, as described above.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each carrier must be “acceptable” in the sense of being compatible with the other ingredients of the formulation and not injurious to the patient.

Some examples of materials which may serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salt” in this respect, refers to the relatively non-toxic, inorganic and organic acid addition salts of compounds of the present invention. These salts may be prepared in situ during the final isolation and purification of the compounds of the invention, or by separately reacting a purified compound of the invention in its free base form with a suitable organic or inorganic acid, and isolating the salt thus formed.

Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (see, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaccutically acceptable bases. The term “pharmaceutically acceptable salt” in these instances refers to the relatively non-toxic, inorganic and organic base addition salts of compounds of the present invention. These salts may likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts, and the like.

Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants may also be present in the compositions.

Examples of pharmaceutically acceptable antioxidants, which may also be present in formulations of therapeutic compounds of the invention, include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal or parenteral administration. The formulations may conveniently be presented in unit dosage form and may be prepared by any methods well known in the art of pharmacy. The amount of active ingredient which may be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules, and the like), the active ingredient is mixed with one or more pharmaceutically acceptable carriers, such as sodium citrate or dicalcium phosphate, or any of the following: fillers or extenders, such as starches, lactose, sucrose, glucose, mannitol, or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents.

In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes or microspheres.

They may be sterilized by, for example, filtration through a bacteria-retaining filter, or by incorporating sterilizing agents in the form of sterile solid compositions which may be dissolved in sterile water, or some other sterile injectable medium immediately before use.

These compositions may also optionally contain opacifying agents and may be of a composition that they release the active ingredient(s) only, or preferentially, in a certain portion of the gastrointestinal tract, optionally, in a delayed manner. Examples of embedding compositions which may be used include polymeric substances and waxes. The active ingredient may also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert dilutents, the oral compositions may also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a compound of this invention, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms may be made by dissolving or dispersing the compound in the proper medium. Absorption enhancers may also be used to increase the flux of the compound across the skin. The rate of such flux may be controlled by either providing a rate controlling membrane or dispersing the active compound in a polymer matrix or gel.

Ophthalmic formulations, eye ointments, powders, solutions and the like, are also within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration,” and “administered peripherally” as used herein mean the administration of a compound, drug or other material other than directly into the central nervous system, such that it enters the patient's system and, thus, is subject to accumulation and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well known in the medical arts.

A physician or veterinarian having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

While it is possible for a compound of the present invention to be administered alone, it is preferable to administer the compound as a pharmaceutical composition.

The regimen of administration may affect what constitutes an effective amount. The fatty acid or triglyceride accumulation modulating, e.g., inhibiting, may be administered to the subject either prior to or after the onset of, for example, obesity. Further, several divided dosages, as well as staggered dosages, may be administered daily or sequentially, or the dose may be continuously infused, or may be a bolus injection. Further, the dosages of the fatty acid or triglyceride accumulation modulating, e.g., inhibiting, compound(s) may be proportionally increased or decreased as indicated by the exigencies of the therapeutic or prophylactic situation.

The present invention also relates to pharmaceutical compositions comprising an effective amount of a compound of any of the compounds described herein, in combination with a second agent. For example the second agent is a weight-reducing or appetite suppressing agent or a chemotherapeutic agent. Pharmaceutical compositions of the invention may further comprise a pharmaceutically acceptable carrier. The invention also relates to a packaged composition for treatment of a disease or condition with any compound described herein, comprising said compound and directions for using said compound for treating said disease according to said method. Such a packaged composition may be used for the treatment or prevention of AIDS, diabetes, coronary disease, lipodystrophy, hypertension, cachexia, anorexia nervosa, bulemia nervosa, hyperinsulinemia, stroke, congestive heart failure, gall stones, gout, hyperlipiedemia, hypercholesterolemia, atherosclerosis or arteriosclerosis, or metabolic syndrome.

The contents of all references and published patents and patent applications cited throughout the application are hereby incorporated by reference.

### EXAMPLES

This invention is further illustrated by the following examples which should not be construed as limiting.

Protocol for High Throughput Screening of Compound Efficacy on Human Preadipocytes

Up to five cell strains of banked primary human subcutaneous preadipocytes were used for high throughput screening. Cells were grown in tissue culture flasks, maintained under standard incubation conditions (5% carbon dioxide, 37° C.) and split evenly when 100% confluent into two new tissue culture flasks (one cell division) in growth medium. Cells were split up to four times to produce enough cells for screening.

Two to five days before the experiment, adhered cells were detached with trypsin/EDTA, combined and seeded into 384-well plates at 100% confluency. On day zero, cells were incubated with growth medium to induce differentiation of preadipocytes into adipocytes (characterized by cell rounding, formation of triglyceride droplets, etc.). Growth medium was changed every three days. On day six, cells were incubated in triplicate with test compounds (supplied by ComGenex International, Inc., South San Francisco, USA) at various concentrations.

Compounds were first diluted in a phosphate-buffered saline solution with 0.1% fatty-acid free bovine serum albumin (BSA, to aid compound suspension), and then added to cells. Negative control cells were treated with DMSO, the solvent used for initially dissolving compounds, at 0.1% final concentration. Positive control cells were treated with carbonyl cyanide p-(trifluoromethoxy)-phenylhydrazone (FCCP, a potent uncoupler of oxidative phosphorylation in mitochondria).

On day 9, a fluorescent fatty acid probe, namely 4,4-difluoro-5-methyl-4-bora-3a,4a-diaza-s-indacene-3-dodecanoic acid (C₁-BODIPY™ 500/510 C₁₂, D-3823 available from Molecular Probes, Eugene, Oreg., USA) (“FA*” herein) was diluted into fatty acid buffer plus BSA (FAB+). Cell plates were prewashed with FAB+, and then FA* was added. Cells were incubated with FA* for four hours, and then postwashed with FAB+ to remove unincorporated FA*. Cellular fluorescence of triglyceride droplets that have incorporated FA* was measured on a microplate reader.

Efficacy of compounds on inhibiting FA* accumulation was determined by the following calculation:

% Efficacy=100-(sample fluorescence/negative control fluorescence*100)

Subsequent determination of compound toxicity was measured by incubating cells with Alamar Blue (an indicator of cellular viability, purchased from BioSource International Inc., Camarillo, Calif., USA) for three to four hours before measuring fluorescence of the reduced compound. Toxicity of compounds was determined by the following calculation:

% Toxicity=100-(sample fluorescence/negative control fluorescence*100)

A control compound capable of inhibiting FA* accumulation in differentiating adipocytes without being toxic is FCCP (C2920 from Sigma Chemical, Carbonyl cyanide p-(trifluoromethoxy)phenylhydrazone). FCCP is a protonophore (H+ionophore) and uncoupler of oxidative phosphorylation in mitochodria. It is capable of depolarizing plasma membrane and mitochondrial membrane and mimics the effect of the glutamate agonist, N-methyl-D-aspartate (NMDA), on mitochondrial superoxide production (see e.g., Tretter et al. (1998) Mol. Pharmacol. 53:734-741; Smith et al. (1999) Pflugers Arch. 437:577-588; Buckler and Vaughan-Jones (1998) J. Physiol. (Lond) 513:819-833; and Sengpiel et al. (1998) Eur. J. Neurosci. 10:1903-1910).

| FCCP: | 0 uM | 0.3 uM | 1 uM | 3 uM |
|---|---|---|---|---|
| % Efficacy: | 0 | 42 | 79 | 90 |
| % Toxicity: | 0 | −1 | −4 | 5 |

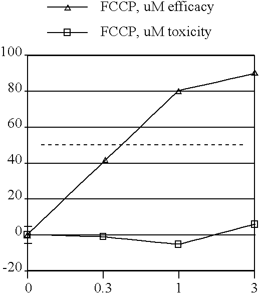

At 10 μM, FCCP shows 100% toxicity as well as efficacy.

Test compounds were screened and assayed as outlined above. Compounds were classified as “inhibitors” or “stimulators” depending on whether they enhanced or suppressed, respectively, the rate of fatty acid accumulation into preadipocyte cells. Examples of the relative IC₅₀ values (from “++++” to “+”) for several inhibitors of the invention are presented in Table I below (the corresponding chemical structures may be found in the accompanying Drawings). Each of the compounds had a negligible “toxicity” according to the assay above.

TABLE I
Relative Efficacy of Some Exemplary Compounds of the Invention

| Compound ID No. | CGX ID No. | Relative Efficacy | Compound ID No. | CGX ID No. | Relative Efficacy |
|---|---|---|---|---|---|
| AGX-0003 | CGX-0419044 | ++++ | AGX-0081 | CGX-0447263 | +++ |
| AGX-0004 | CGX-0384024 | ++++ | AGX-0085 | CGX-0438927 | +++ |
| AGX-0005 | CGX-0386138 | ++++ | AGX-0089 | CGX-0480303 | +++ |
| AGX-0006 | CGX-0397636 | ++++ | AGX-0093 | CGX-0447278 | +++ |
| AGX-0009 | CGX-0487670 | ++++ | AGX-0102 | CGX-0322278 | +++ |
| AGX-0019 | CGX-0510288 | ++++ | AGX-0016 | CGX-0498043 | ++ |
| AGX-0020 | CGX-0510291 | ++++ | AGX-0022 | CGX-0335604 | ++ |
| AGX-0029 | CGX-0398095 | ++++ | AGX-0024 | CGX-0328203 | ++ |
| AGX-0032 | CGX-0416349 | ++++ | AGX-0027 | CGX-0429466 | ++ |
| AGX-0033 | CGX-0386778 | ++++ | AGX-0031 | CGX-0421170 | ++ |
| AGX-0040 | CGX-0445808 | ++++ | AGX-0036 | CGX-0428371 | ++ |
| AGX-0042 | CGX-0445478 | ++++ | AGX-0039 | CGX-0445522 | ++ |
| AGX-0059 | CGX-0482232 | ++++ | AGX-0043 | CGX-0436221 | ++ |
| AGX-0060 | CGX-0495385 | ++++ | AGX-0047 | CGX-0405862 | ++ |
| AGX-0065 | CGX-0437610 | ++++ | AGX-0050 | CGX-0449554 | ++ |
| AGX-0069 | CGX-0436493 | ++++ | AGX-0056 | CGX-0417165 | ++ |
| AGX-0072 | CGX-0437178 | ++++ | AGX-0057 | CGX-0472190 | ++ |
| AGX-0073 | CGX-0437226 | ++++ | AGX-0058 | CGX-0486382 | ++ |
| AGX-0076 | CGX-0425850 | ++++ | AGX-0070 | CGX-0437170 | ++ |
| AGX-0083 | CGX-0446603 | ++++ | AGX-0074 | CGX-0343332 | ++ |
| AGX-0092 | CGX-0436549 | ++++ | AGX-0084 | CGX-0446618 | ++ |
| AGX-0094 | CGX-0405573 | ++++ | AGX-0086 | CGX-0426665 | ++ |
| AGX-0101 | CGX-0420134 | ++++ | AGX-0087 | CGX-0426673 | ++ |
| AGX-0017 | CGX-0500292 | +++ | AGX-0090 | CGX-0461890 | ++ |
| AGX-0018 | CGX-0520777 | +++ | AGX-0095 | CGX-0471359 | ++ |
| AGX-0021 | CGX-0509517 | +++ | AGX-0098 | CGX-0391650 | ++ |
| AGX-0030 | CGX-0420994 | +++ | AGX-0099 | CGX-0412511 | ++ |
| AGX-0048 | CGX-0332396 | +++ | AGX-0014 | CGX-0466396 | + |
| AGX-0051 | CGX-0405309 | +++ | AGX-0015 | CGX-0513066 | + |
| AGX-0053 | CGX-0447290 | +++ | AGX-0023 | CGX-0325317 | + |
| AGX-0054 | CGX-0433534 | +++ | AGX-0034 | CGX-0369189 | + |
| AGX-0055 | CGX-0441646 | +++ | AGX-0035 | CGX-0418441 | + |
| AGX-0062 | CGX-0493337 | +++ | AGX-0049 | CGX-0425654 | + |
| AGX-0088 | CGX-0458723 | +++ | AGX-0063 | CGX-0378337 | + |
| AGX-0007 | CGX-0345648 | +++ | AGX-0064 | CGX-0409473 | + |
| AGX-0008 | CGX-0407528 | +++ | AGX-0067 | CGX-0404852 | + |
| AGX-0010 | CGX-0471298 | +++ | AGX-0077 | CGX-0433334 | + |
| AGX-0013 | CGX-0466508 | +++ | AGX-0082 | CGX-0447286 | + |
| AGX-0025 | CGX-0334477 | +++ | AGX-0096 | CGX-0348074 | + |
| AGX-0028 | CGX-0459150 | +++ | AGX-0097 | CGX-0367372 | + |
| AGX-0037 | CGX-0380173 | +++ | AGX-0100 | CGX-0420120 | + |
| AGX-0041 | CGX-0366708 | +++ | AGX-0103 | CGX-0344401 | + |
| AGX-0044 | CGX-0445566 | +++ | AGX-0026 | CGX-0433466 | + |
| AGX-0045 | CGX-0429934 | +++ | AGX-0011 | CGX-0453674 | + |
| AGX-0046 | CGX-0433449 | +++ | AGX-0012 | CGX-0466395 | + |
| AGX-0052 | CGX-0385812 | +++ | AGX-0001 | CGX-0309650 | + |
| AGX-0061 | CGX-0495393 | +++ | AGX-0002 | CGX-0312280 | + |
| AGX-0068 | CGX-0435360 | +++ | AGX-0038 | CGX-0368082 | + |
| AGX-0071 | CGX-0437090 | +++ | AGX-0091 | CGX-0461907 | + |
| AGX-0075 | CGX-0410920 | +++ | AGX-0066 | CGX-0392506 | + |
| AGX-0078 | CGX-0437141 | +++ | | | |
| AGX-0079 | CGX-0445498 | +++ | | | |
| AGX-0080 | CGX-0442365 | +++ | | | |

The most preferred inhibitors have compound numbers AGX-0034, AGX-0020, AGX-0088, AGX-0018, AGX-0042, AGX-0099, AGX-0013, AGX-0025, and AGX-0008 because of their IC₅₀ values, as well as their stability and toxicity profiles.

Stimulators of Fatty Acid Accumulation

| | | |
|---|---|---|
| AGX-0111 CGX-0389934 | 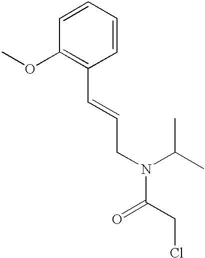 | +++ |
| AGX-0112 CGX-0438827 | 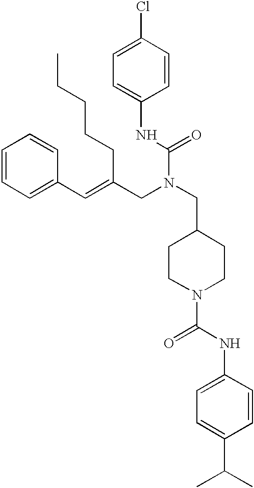 | +++ |
| AGX-0113 CGX-0331964 | 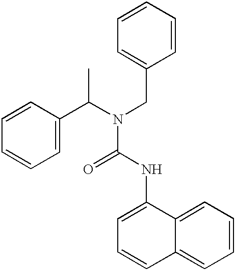 | ++ |

Compounds for use in the instant invention may be purchased from ComGenex International, Inc. (South San Francisco, USA) as representative samples of various combinatorial libraries, and could be isolated from said library by the screening methods described herein. In the Table and the accompanying Drawings, ComGenex's compounds identification numbers (“CGX” numbers) are provided for direct ordering of the compounds.

Alternatively, compounds for use according to the invention may be synthesized according to art recognized methods: For example, compounds of Formula I may be synthesized, in general, by reacting an appropriately protected arylaminocarbonoyl chloride or activated ester with a suitably protected unsubstituted amine-containing group Z, followed by deprotection and substitution of said amine-containing group Z by, for example, alkylation or acetylation. Similarly, compounds according to Formula II may be synthesized, in general, by reacting an appropriately protected arylamine with an acetylating reagent, for example an acid chloride or an activated ester, followed by deprotection.

Equivalents

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference in their entirety.

We claim:

1. A method of modulating the accumulation of a fatty acid or triglyceride in a cell, comprising a step of contacting said cell with a compound, wherein said compound comprises a substituted or unsubstituted aryl group and an amide, sulfonamide, or ureylene group, such that modulation of said fatty acid or triglyceride accumulation occurs.

2. The method according to claim 1, wherein said modulating property is an increase in the accumulation of fatty acids or triglycerides by cells.

3. The method according to claim 1, wherein said modulating property is a decrease in the accumulation of fatty acids or triglycerides by cells.

4. The method according to any one of the foregoing claims, wherein modulation of said fatty acid or triglyceride uptake is a means of treating or preventing a disease or condition in a subject.

5. The method according to claim 4, wherein said subject is affected with said disease or condition, has a susceptibility thereto, or has a medical history thereof.

6. The method according to claim 4, wherein said subject is a human.

7. The method according to claim 6, wherein said subject is overweight.

8. The method according to claim 6, wherein said subject is underweight.

9. The method according to claim 6, wherein said subject is obese.

10. The method according to claim 6, wherein said subject has a Body Mass Index of less than about 25.

11. The method according to claim 6, wherein said subject has a Body Mass Index of less than about 20.

12. The method according to claim 6, wherein said subject has a Body Mass Index of less than about 18.

13. The method according to claim 6, wherein said subject has a Body Mass Index of greater than about 20.

14. The method according to claim 6, wherein said subject has a Body Mass Index of greater than about 25.

15. The method according to claim 6, wherein said subject has a Body Mass Index of greater than about 30.

16. The method according to claim 6, wherein said subject has a Body Mass Index of greater than about 32.

17. The method according to claim 6, wherein said subject has a Body Mass Index of greater than about 34.

18. The method according to claim 6, wherein said subject has a Body Mass Index of greater than about 36.

19. The method according to claim 6, wherein said subject has a Body Mass Index of greater than about 38.

20. The method according to claim 6, wherein said subject has a Body Mass Index of greater than about 40.

21. The method according to claim 5, wherein said disease or condition is cancer, AIDS, diabetes, coronary disease, lipodystrophy, hypertension, cachexia, anorexia nervosa, bulemia nervosa, hyperinsulinemia, stroke, congestive heart failure, gall stones, gout, hyperlipiedemia, hypercholesterolemia, atherosclerosis or arteriosclerosis, or metabolic syndrome; a susceptibility thereto, a medical history thereof, or a pathological consequence thereof.

22. The method according to claim 1, wherein said compound is selected according to the following Formula: 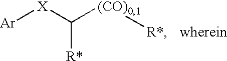
Ar is a substituted or unsubstituted aryl group,
X is L or SO₂,
R* is an organic moiety, and
L is a linking group,
and pharmaceutically acceptable salts thereof.

23. The method according to claim 1, wherein said compound is selected according to the following Formula: 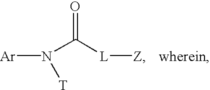
Ar is a substituted or unsubstituted aryl group,
T is a hydrogen or a C₁₋₅ straight or branched chain alkyl group,
L is a linking group, and
Z is a substituted alkyleneamine or amine derivative,
and pharmaceutically acceptable salts thereof.

24. The method according to claim 1, wherein said compound is selected according to the following Formula: 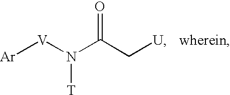
Ar is a substituted or unsubstituted aryl group,
V is a substituted or unsubstituted C₁₋₆ straight or branched chain alkylene group, or a substituted or unsubstituted C₂₋₆ straight or branched chain alkenylene or alkynylene group,
T is a hydrogen or a C₁₋₅ straight or branched chain alkyl group, and
U is a halogen, or a C(halogen)₃, CH(halogen)₂, CH₂(halogen), alkyl, or nitro group,
and pharmaceutically acceptable salts thereof.

25. The method according to claim 23, wherein Z is selected from the group consisting of 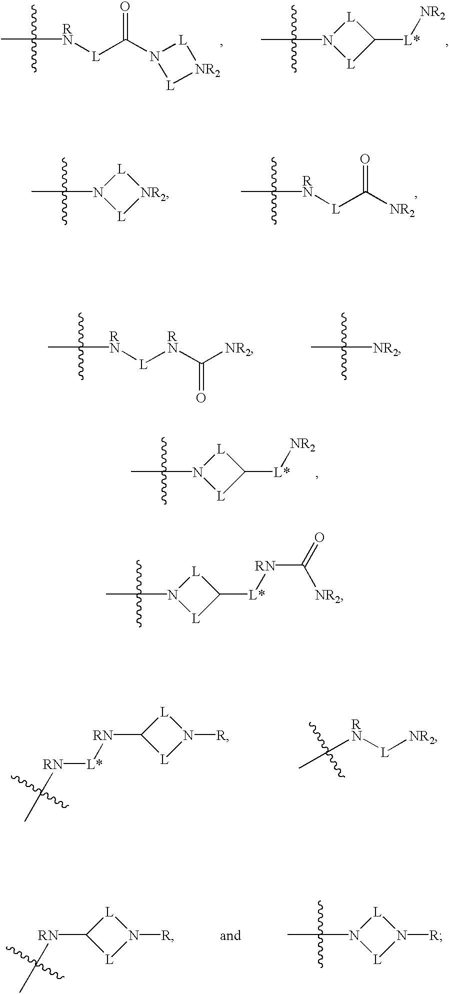
wherein L is a linking group,
L* is L-(CO)_{0,1} or (CO)_{0,1}-L, and
each R is an organic moiety,
provided that at least one L or L* group in a molecule, if present, is not a direct bond, and at least one R group per molecule is not a hydrogen.

26. The method according to claim 1, wherein said compound has the structure 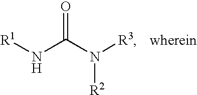
each R group is independently selected from the group consisting of a hydrogen atom, a substituted or unsubstituted straight or branched alkyl, substituted or unsubstituted cycloalkyl, substituted or unsubstituted alkenyl, substituted or unsubstituted alkynyl, substituted or unsubstituted heterocyclic, substituted or unsubstituted carbocyclic, substituted or unsubstituted aryl, substituted or unsubstituted aralkyl, substituted or unsubstituted aryloxyalkyl, substituted or unsubstituted arylacetamidoyl, substituted or unsubstituted alkylaryl, substituted or unsubstituted heteroaralkyl, substituted or unsubstituted alkylcarbonyl, substituted or unsubstituted arylcarbonyl, substituted or unsubstituted heteroarylcarbonoyl, or substituted or unsubstituted heteroaryl group;
or two R groups taken together when bound to the same nitrogen atom form a substituted or unsubstituted heterocyclic ring; or
(CR′R″)₁₋₁₂H, (CR′R″)₀₋₃NR′R″, (CR′R″)₀₋₃CN, (CR′R″)₀₋₃NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, and (CR′R″)₀₋₃OR′;
wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group;
and pharmaceutically acceptable salts thereof.

27. The method according to claim 26, wherein each R group is independently selected from the group consisting of a hydrogen atom, a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted aryl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; or
two R groups taken together when bound to the same nitrogen atom form a substituted or unsubstituted morpholine or piperidine ring; or
(CR′R″)₀₋₃NH₂, (CR′R″)₀₋₃CN, (CR′R″)₀₋₃NO₂, (CR′R″)₀₋₃CF₃, (CR′R″)₀₋₃CHF₂, (CR′R″)₀₋₃CH₂F, (CR′R″)₀₋₃CONH₂, (CR′R″)₀₋₃S(O)₁₋₂NH₂, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂H, and (CR′R″)₀₋₃OH;
wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

28. The method according to claim 26, wherein each R group is independently selected from the group consisting of a hydrogen atom, a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted phenyl or naphthyl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; or
two R groups taken together when bound to the same nitrogen atom form a substituted or unsubstituted morpholine or piperidine ring; or
(CH₂)₁₋₃NH₂, (CH₂)₁₋₃CN, (CH₂)₁₋₃NO₂, (CH₂)₁₋₃CF₃, (CH₂)₁₋₃CHF₂, (CH₂)₁₋₃CH₂F, (CH₂)₁₋₃CONH₂, (CH₂)₁₋₃S(O)₁₋₂NH₂, (CH₂)₁₋₃CHO, (CH₂)₁₋₃O(CH₂)₁₋₃H, (CH₂)₁₋₃S(O)₀₋₂H, (CH₂)₁₋₃O(CH₂)₁₋₃H, (CH₂)₁₋₃COH, (CH₂)₁₋₃CO₂H, and (CH₂)₁₋₃OH.

29. The method according to claim 26, wherein R¹ is a substituted phenyl group.

30. The method according to claim 1, wherein said compound has the structure 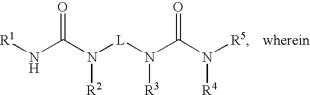
each R group is as defined in claim 26, and L is a linking group.

31. The method according to claim 30, wherein R¹ is a substituted phenyl group.

32. The method according to claim 30, wherein R² is hydrogen.

33. The method according to claim 30, wherein R⁴ is hydrogen.

34. The method according to claim 30, wherein R⁵ is a substituted phenyl group, a naphthyl group, or a cycloalkyl group.

35. The method according to claim 30, wherein L and R² are taken together to form a cyclic alkylene group according to the following structure

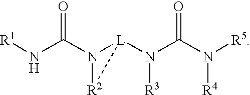

36. The method according to claim 35, wherein L and R² are taken together to form a cyclic alkylene group according to the following structure

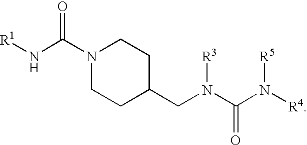

37. The method according to claim 1, wherein said compound has the structure 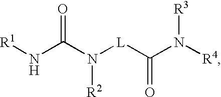
wherein each R group is as defined in claim 26, and L is a linking group.

38. The method according to claim 37, wherein R¹ is a substituted alkyl group.

39. The method according to claim 37, wherein R¹ is a substituted aralkyl group.

40. The method according to claim 37, wherein R³ and R⁴ are taken together to form a heterocyclic moiety.

41. The method according to claim 40, wherein the compound has the following structure 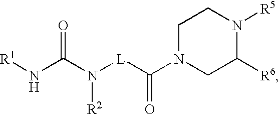
wherein L is a linking group, R⁵ is an R group as defined in claim 26, and R⁶ is hydrogen or a an alkyl group.

42. The method according to claim 41, wherein R¹ is a substituted alkyl group.

43. The method according to claim 41, wherein R¹ is a substituted aralkyl group.

44. The method according to claim 41, wherein R⁶ is a methyl group.

45. The method according to claim 1, wherein said compound has the structure 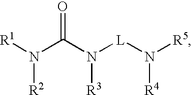
wherein each R group is as defined in claim 26, and L is a linking group.

46. The method according to claim 45, wherein R¹ is a substituted phenyl group.

47. The method according to claim 45, wherein R² is a hydrogen.

48. The method according to claim 45, wherein L and R³ are taken together to form a cyclic alkylene group according to the following structure

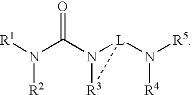

49. The method according to claim 48, wherein L and R³ are taken together to form a cyclic alkylene group according to the following structure

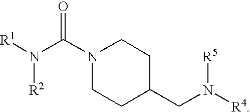

50. The method according to claim 49, wherein R¹ is a substituted phenyl group.

51. The method according to claim 49, wherein R² is a hydrogen.

52. The method according to claim 1, wherein said compound has the following structure 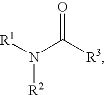
wherein each R group is as defined in claim 26.

53. The method according to claim 52, wherein R¹ and R² are independently a substituted or unsubstituted C₁-C₆ alkyl group or a substituted or unsubstituted C₂-C₆ alkenyl group.

54. The method according to claim 52, wherein R³ is a substituted or unsubstituted C₁-C₆ alkyl group or a substituted or unsubstituted phenyl or naphthyl group.

55. The method according to claim 52, wherein R¹ and R² are taken together to form a heterocyclic moiety.

56. The method according to claim 55, wherein the compound has the following structure 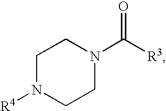
wherein R⁴ is an R group as defined in claim 26.

57. The method according to claim 1, wherein said compound has the structure 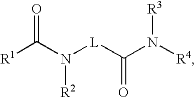
wherein each R group is as defined in claim 26, and L is a linking group.

58. The method according to claim 57, wherein R³ and R⁴ are taken together to form a heterocyclic moiety.

59. The method according to claim 58, wherein the compound has the structure 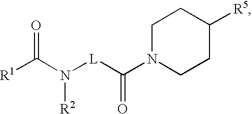
wherein R⁵ is an R group as defined in claim 26.

60. The method according to claim 58, wherein the compound has the structure 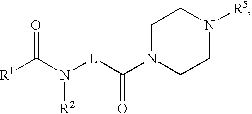
wherein R⁵ is an R group as defined in claim 26.

61. The method according to claim 1, wherein said compound has the structure 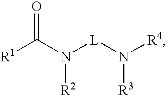
wherein each R group is as defined in claim 26, and L is a linking group.

62. The method according to claim 61, wherein R¹ is a substituted phenyl group.

63. The method according to claim 61, wherein R² is a substituted or unsubstituted C₁-C₆ alkyl group.

64. The method according to claim 61, wherein R³ and R⁴ are independently a substituted or unsubstituted C₁-C₆ alkyl group.

65. The method according to claim 61, wherein R³ and L are taken together to form a cyclic alkylene group according to the following structure

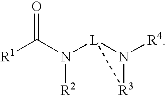

66. The method according to claim 65, wherein R² and L are taken together to form a cyclic alkylene group according to the following structure

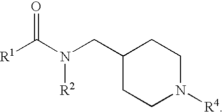

67. The method according to claim 66, wherein R¹ is a substituted phenyl group.

68. The method according to claim 66, wherein R² is a substituted or unsubstituted C₁-C₆ alkyl group.

69. The method according to claim 65, wherein R³ and R⁴ are taken together to form a heterocyclic moiety.

70. The method according to claim 69, wherein the compound has the structure 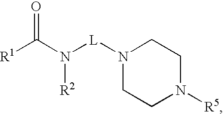
wherein R⁵ is an R group as defined in claim 26.

71. The method according to claim 1, wherein said compound has the structure 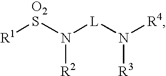
wherein each R group is as defined in claim 26, and L is a linking group.

72. The method according to claim 71, wherein R¹ is a substituted phenyl group or a naphthyl group.

73. The method according to claim 71, wherein R² is a substituted or unsubstituted C₁-C₁₂ alkyl group or a substituted or unsubstituted C₂-C₁₂ alkenyl group.

74. The method according to claim 71, wherein R³ and R⁴ are independently a substituted or unsubstituted C₁-C₁₂ alkyl group or a substituted or unsubstituted C₂-C₁₂ alkenyl group.

75. The method according to claim 71, wherein R³ and R⁴ are taken together to form a heterocyclic moiety.

76. The method according to claim 75, wherein the compound has the following structure 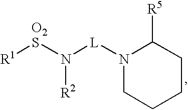
wherein R⁵ is hydrogen or a an alkyl group.

77. The method according to claim 76, wherein R⁵ is a methyl group.

78. The method according to claim 76, wherein R¹ is a substituted phenyl group or a naphthyl group.

79. The method according to claim 76, wherein R² is a substituted or unsubstituted C₁-C₁₂ alkyl group or a substituted or unsubstituted C₂-C₁₂ alkenyl group.

80. The method according to any one of the foregoing claims, wherein R¹ is a naphthyl group or an Ar group, wherein said Ar group is selected from the group consisting of 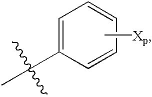
p is an integer from zero to five inclusive,
X is selected from the group consisting of a substituted or unsubstituted straight or branched alkyl, substituted or unsubstituted cycloalkyl, substituted or unsubstituted alkenyl, substituted or unsubstituted alkynyl, substituted or unsubstituted heterocyclic, substituted or unsubstituted carbocyclic, substituted or unsubstituted aryl, substituted or unsubstituted aralkyl, substituted or unsubstituted aryloxyalkyl, substituted or unsubstituted arylacetamidoyl, substituted or unsubstituted alkylaryl, substituted or unsubstituted heteroaralkyl, substituted or unsubstituted alkylcarbonyl, substituted or unsubstituted arylcarbonyl, substituted or unsubstituted heteroarylcarbonoyl, or substituted or unsubstituted heteroaryl group; or
(CR′R″)₁₋₁₂H, (CR′R″)₀₋₃NR′R″, (CR′R″)₀₋₃CN, (CR′R″)₀₋₃NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, and (CR′R″)₀₋₃OR′;
wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

81. The method according to claim 80, wherein X is independently selected from the group consisting of a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted aryl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; or
(CR′R″)₀₋₃NH₂, (CR′R″)₀₋₃CN, (CR′R″)₀₋₃NO₂, (CR′R″)₀₋₃CF₃, (CR′R″)₀₋₃CHF₂, (CR′R″)₀₋₃CH₂F, (CR′R″)₀₋₃CONH₂, (CR′R″)₀₋₃S(O)₁₋₂NH₂, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂H, and (CR′R″)₀₋₃OH;
wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

82. The method according to claim 80, wherein X is selected from the group consisting of a substituted or unsubstituted straight or branched C₁-C₁₀ alkyl, substituted or unsubstituted C₃-C₈ cycloalkyl, substituted or unsubstituted C₂-C₁₀ alkenyl, substituted or unsubstituted C₂-C₁₀ alkynyl, substituted or unsubstituted heterocyclic, substituted or unsubstituted carbocyclic, substituted or unsubstituted phenyl or naphthyl, substituted or unsubstituted aryl-(C₁-C₁₀ alkyl), substituted or unsubstituted aryloxy-(C₁-C₁₀ alkyl), substituted or unsubstituted arylacetamidoyl, substituted or unsubstituted (C₁-C₁₀ alkyl)-aryl, substituted or unsubstituted heteroaryl-(C₁-C₁₀ alkyl), substituted or unsubstituted (C₁-C₁₀ alkyl)carbonyl, substituted or unsubstituted arylcarbonyl, substituted or unsubstituted heteroarylcarbonoyl, or substituted or unsubstituted heteroaryl group; or
(CH₂)₁₋₃NH₂, (CH₂)₁₋₃CN, (CH₂)₁₋₃NO₂, (CH₂)₁₋₃CF₃, (CH₂)₁₋₃CHF₂, (CH₂)₁₋₃CH₂F, (CH₂)₁₋₃CONH₂, (CH₂)₁₋₃S(O)₁₋₂NH₂, (CH₂)₁₋₃CHO, (CH₂)₁₋₃O(CH₂)₁₋₃H, (CH₂)₁₋₃S(O)₀₋₂H, (CH₂)₁₋₃O(CH₂)₁₋₃H, (CH₂)₁₋₃COH, (CH₂)₁₋₃CO₂H, and (CH₂)₁₋₃OH.

83. The method according to claim 80, wherein p is 1.

84. The method according to claim 80, wherein p is 2.

85. The method according to claim 80, wherein X is a halogen, C(halogen)₃, CH(halogen)₂, CH₂(halogen), alkyl, or nitro group.

86. The method according to claim 80, wherein X is CF₃, CCl₃, CHF₂, CHCl₂, F, Cl, Br, I, NO₂, C₂-C₁₀ n-alkyl group, CN, OCH₃, CH₃, phenoxy, phenyl, OCH₂CH₃, or CH₂CH₃.

87. The method according to claim 80, wherein X is a methyl, ethyl, propyl, butyl, pentyl, hexyl, heptyl, octyl, or nonyl group.

88. The method according to claim 80, wherein p is 1, and X is o-F, o-Me, p-OCH₃, m-F, m-CN, m-CF₃, m-Cl, p-NO₂, p-phenoxy, m-CH₃, p-Cl, p-Br, o-phenyl, p-CF₃, p-ethyl, p-ethoxy, m-Br, or m-NO₂.

89. The method according to claim 80, wherein p is 2, and both X groups are m,m-F₂, m,p-O₂(CH₂), m,p-Cl₂, o,o-(CH₃)₂, or o,p-Cl₂.

90. The method according to claim 80, wherein Ar is

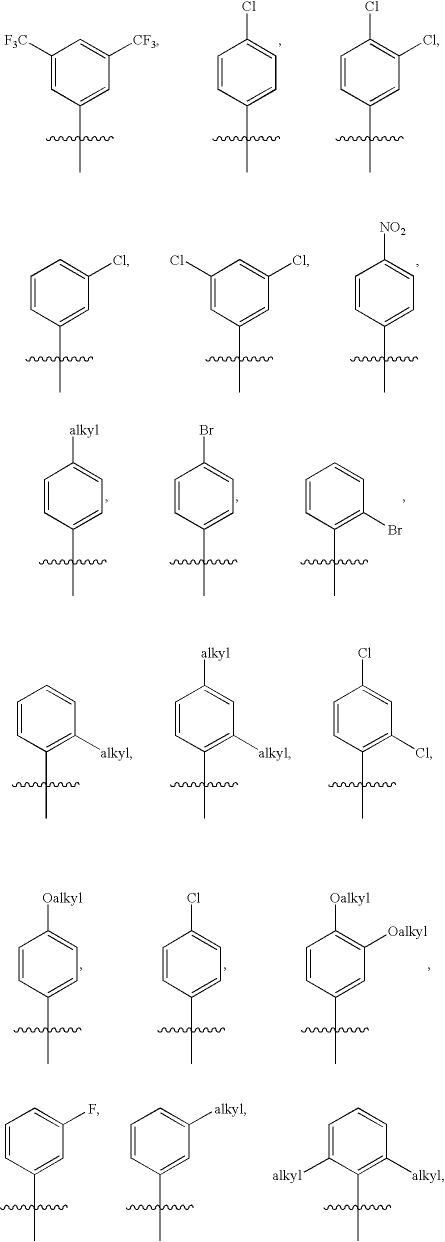

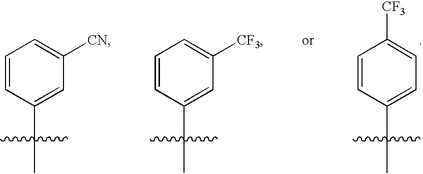

91. The method according to claim 90, wherein said alkyl group is an n-alkyl or iso-alkyl group.

92. The method according to claim 90, wherein said alkyl group is an n-heptyl or iso-propyl group.

93. The method according to any one of the foregoing claims, wherein L is a linking group of less than about 250 molecular weight.

94. The method according to claim 93, wherein L is a linking group of less than about 75 molecular weight.

95. The method according to claim 93, wherein L is an unsubstituted C₁-C₆ alkylene group.

96. The method according to claim 93, wherein L is a substituted C₁-C₆ alkylene group.

97. The method according to claim 93, wherein L is a bond, (CR^{a}R^{b})ₙ, CR^{a}OR^{b}(CR^{c}R^{d})ₙ, CR^{a}SH(CR^{c}R^{d})ₙ, CR^{a}NR^{b}R^{c}(CR^{d}R^{e})ₙ, (CR^{a}R^{b})ₙO(CR^{c}R^{d})ₙ, wherein each n is independently either 0, 1, 2, or 3, and R^{a}, R^{b}, R^{c}, R^{d}, and R^{e} are each independently hydrogen, a substituted or unsubstituted C₁-C₅ branched or straight chain alkyl or alkoxy, C₂-C₅ branched or straight chain alkenyl, aryloxycarbonyl, arylaminocarbonyl, arylalkyl, acyl, aryl, or C₃-C₈ ring group.

98. The method according to claim 93, wherein L is a (CR^{a}R^{b})ₙ wherein n is either 0, 1, 2, or 3, and R^{a} and R^{b} are each independently hydrogen, a C₁-C₅ branched or straight chain alkyl or alkoxy, arylalkyl, aryl, or a C₃-C₈ cycloalkyl group.

99. The method according to claim 98, wherein R^{a} and R^{b} are each independently hydrogen, methyl, benzyl, phenylethyl, sec-phenylethyl, iso-butyl, or iso-propyl group.

100. The method according to claim 98, wherein L is (CH₂)₂, (CH₂)₃, or (CH₂)₄.

101. The method according to any one of the foregoing claims, wherein said R group is a substituted or unsubstituted branched, bicyclic, cyclic, or unbranched C₁-C₂₀ alkyl group or a substituted or unsubstituted branched, bicyclic, cyclic, or unbranched C₂-C₂₀ alkylene group.

102. The method according to any one of the foregoing claims, wherein said R group is an iso-propyl, methyl, iso-butyl, 2-benzylideneheptyl, sec-butyl, cyclohexyl, cyclopropyl, 2-(N-morpholinyl)-ethyl, a sec-phenylethyl or phenylethyl group.

103. The method according to any one of the foregoing claims, wherein said R group is 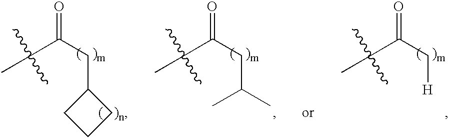
wherein each m is an integer from 1 to 8 inclusive, and each n is an integer from 0 to 5 inclusive.

104. The method according to any one of the foregoing claims, wherein said R group is 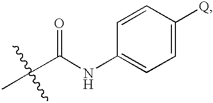
wherein Q is a halogen, hydrogen, or a C₁-C₅ alkyl, O(C₁-C₅ alkyl), or benzyloxy group.

105. The method according to any one of the foregoing claims, wherein said R group is a cyclopropyl or cyclohexyl group.

106. The method according to any one of the foregoing claims, wherein said R group is 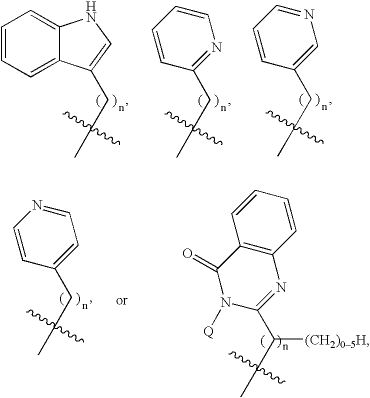
wherein n is an integer from 1 to 3 inclusive, and Q is a C₁-C₅ alkyl, C₂-C₅ alkenyl, or C₂-C₅ alkynyl group.

107. The method according to any one of the foregoing claims, wherein said R group is 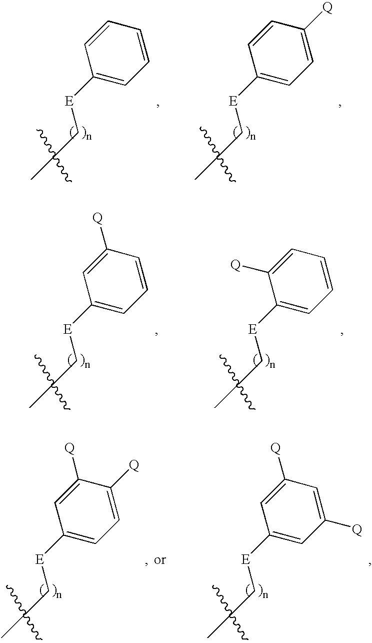
wherein n is an integer from 0 to 5 inclusive, and Q is a C₁-C₅ alkyl, O(C₁-C₅ alkyl), or benzyloxy group, or Q is a halogen, and E is a direct bond or an oxygen atom.

108. The method according to any one of the foregoing claims, wherein said R group is a benzoyl,

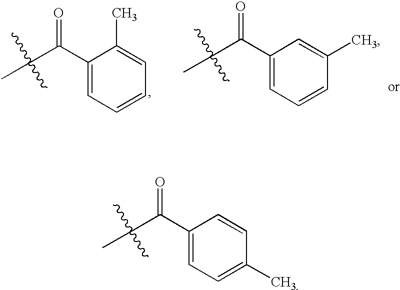

109. The method according to any one of the foregoing claims, wherein said R group is 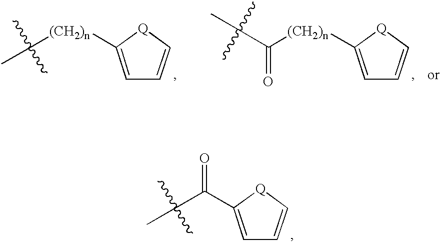
wherein Q is NH, O, or S, and n is an integer from 0 to 6.

110. The method according to any one of the foregoing claims, wherein said R group is 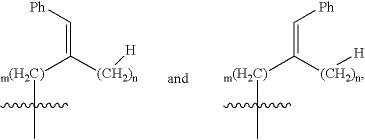
wherein m is an integer from 0 to 3, and n is an integer from 1 to 7.

111. The method according to any one of the foregoing claims, wherein said R group is (CR′R″)₀₋₃CH(phenyl)₂; wherein R′ and R″ are each independently hydrogen, a C₁-C₅ alkyl, C₂-C₅ alkenyl, C₂-C₅ alkynyl.

112. The method according to any one of the foregoing claims, wherein said R group is a naphthyl group, or a partially hydrogenated derivative thereof.

113. The method according to any one of the foregoing claims, wherein said R group is 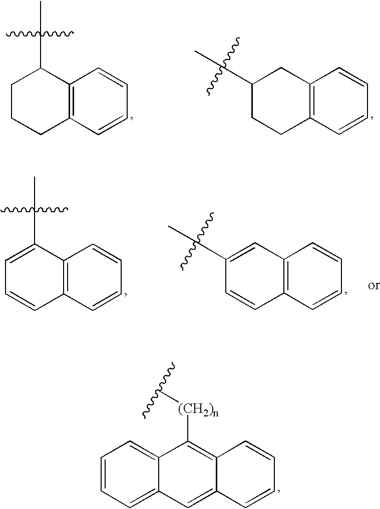
wherein n is an integer from 1 to 4.

114. The method according to any one of the foregoing claims, wherein said two R groups taken together when bound to the same nitrogen atom are 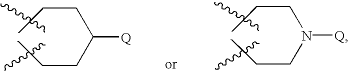
wherein Q is an R group as defined in claim 26.

115. The method according to claim 114, wherein Q is alkyl, aralkyl, heteroaryl group, hydrogen, a benzyl group, or

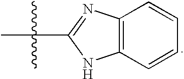

116. The method according to any one of the foregoing claims, wherein said R group is 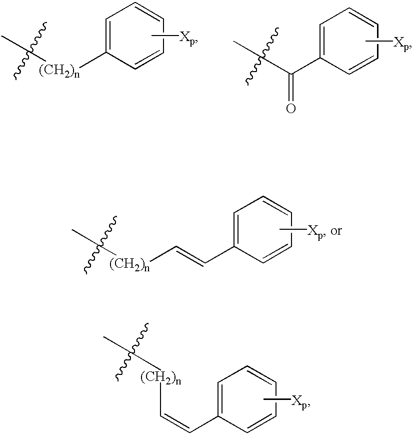
wherein
n is an integer from zero to six inclusive,
p is an integer from zero to five inclusive,
X is selected from the group consisting of a substituted or unsubstituted straight or branched alkyl, substituted or unsubstituted cycloalkyl, substituted or unsubstituted alkenyl, substituted or unsubstituted alkynyl, substituted or unsubstituted heterocyclic, substituted or unsubstituted carbocyclic, substituted or unsubstituted aryl, substituted or unsubstituted aralkyl, substituted or unsubstituted aryloxyalkyl, substituted or unsubstituted arylacetamidoyl, substituted or unsubstituted alkylaryl, substituted or unsubstituted heteroaralkyl, substituted or unsubstituted alkylcarbonyl, substituted or unsubstituted arylcarbonyl, substituted or unsubstituted heteroarylcarbonoyl, or substituted or unsubstituted heteroaryl group; or
(CR′R″)₁₋₁₂H, (CR′R″)₀₋₃NR′R″, (CR′R″)₀₋₃CN, (CR′R″)₀₋₃NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, and (CR′R″)₀₋₃OR′;
wherein each of R′ and R″ is independently hydrogen, a C₁-C₅ alkyl, C₂-C₅ alkenyl, C₂-C₅ alkynyl, aryl-(C₁-C₅ alkyl), or aryl group, or R′ and R″ taken together are a benzylidene group or a —(CH₂)ₙO(CH₂)ₙ— (wherein each n is 1, 2, or 3) group.

117. The method according to any one of the foregoing claims, wherein said R group is 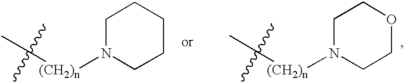
wherein
n is an integer from zero to six inclusive.

118. The method of modulating the accumulation of a fatty acid or triglyceride according to any claim herein, wherein the said compound is selected from those depicted in the accompanying Drawings, and, pharmacautically acceptable salts thereof.

119. The method of according to claim 1, wherein the said compound is AGX-0034, AGX-0020, AGX-0088, AGX-0018, AGX-0042, AGX-0099, AGX-0013, AGX-0025, and AGX-0008.

120. A pharmaceutical composition for use in the method of any claim herein.

121. A prodrug pharmaceutical composition for use in the method of any claim herein.

122. The method of any one of the preceding claims, wherein said compound is administered with a suitable pharmaceutical carrier.

123. A pharmaceutical composition comprising an effective amount of a compound of any of the preceding claims in combination with a second agent.

124. The pharmaceutical composition of claim 123, wherein said second agent is a weight-reducing or appetite suppressing agent.

125. The pharmaceutical composition of claim 123, wherein said second agent is a chemotherapeutic agent.

126. The pharmaceutical composition of any one of the foregoing claims, wherein said pharmaceutical composition further comprises a pharmaceutically acceptable carrier.

127. The pharmaceutical composition of claim 126, wherein said effective amount is effective to treat a lipid metabolism or uptake disorder.

128. The pharmaceutical composition of claim 127, wherein said effective amount is effective to treat obesity.

129. A packaged composition for treatment of a disease or condition with any compound according to any of the foregoing claims, comprising said compound and directions for using said compound for treating said disease according to said method.

130. The packaged composition of claim 129, further comprising a pharmaceutically acceptable carrier.

131. The packaged composition of claim 129, wherein said disease cancer, AIDS, diabetes, coronary disease, lipodystrophy, hypertension, cachexia, anorexia nervosa, bulemia nervosa, hyperinsulinemia, stroke, congestive heart failure, gall stones, gout, hyperlipiedemia, hypercholesterolemia, atherosclerosis or arteriosclerosis, or metabolic syndrome.

132. The packaged composition of any one of claims 129-131, further comprising a second agent.

133. The packaged composition of claim 132, wherein said second agent is a weight-reducing or appetite suppressing agent.

134. The method according to claim 1, wherein said cell is an adipocyte or preadipocyte.

135. The method according to claim 1, wherein said cell is subcutaneous.

136. The method according to claim 1, wherein said cell is visceral.

137. The method according to claim 4, wherein said subject is a companion animal.

138. The method according to claim 137, wherein said companion animal is a dog or cat.

139. The method according to claim 1, wherein modulation of said fatty acid or triglyceride accumulation is a means of producing leaner or fatter livestock.

140. The method according to claim 139, wherein said livestock are pigs, cows, lamb, sheep, or horses.

141. An animal feedstock comprising a chemical according to any Formula herein.
